(19) United States
(12) Patent Application Publication
(10) Pub. No.: US 2019/0119387 A1
(43) Pub. Date: Apr. 25, 2019

(54) INHIBITION OF TGFBETA IN IMMUNOTHERAPY

(71) Applicant: GlaxoSmithKline Intellectual Property Development Limited, Brentford, Middlesex (GB)

(72) Inventor: Sara Jane BRETT, Stevenage (GB)

(21) Appl. No.: 16/091,564

(22) PCT Filed: Apr. 5, 2017
(86) PCT No.: PCT/IB2017/051940

§ 371 Date: Oct. 5, 2018

Related U.S. Application Data
(60) Provisional application No. 62/318,441, filed on Apr. 5, 2016.

(51) Int. Cl. C07K 16/28; C07K 16/22; C07K 14/725; A61K 35/17; C07K 14/705

(52) U.S. Cl. CPC C07K 16/2863; C07K 16/22; C07K 14/7051; A61K 35/17; A61K 2039/505; C07K 14/70521; C07K 2317/76; C07K 2317/622; C07K 14/70517

(57) ABSTRACT

The invention relates to improved compositions and methods for treating diseases, such as cancer, by providing a combination therapy comprising a TGFβ Receptor (TGFβR) antagonist comprising a domain antibody and a cell immunotherapy, in particular where the cell immunotherapy is an immunomodulatory cell expressing a chimeric antigen receptor (CAR) or a modified T cell receptor (TCR). The invention further relates to polynucleotides, expression vectors and immunomodulatory cells comprising the combination therapy, as well as methods of generating said immunomodulatory cells.

### CROSS-REFERENCE TO RELATED APPLICATIONS

This application claims benefit of U.S. Provisional Application No. 62/318,441, filed 5 Apr. 2016, the disclosure of which is incorporated herein by reference in its entirety.

### FIELD OF THE INVENTION

The invention relates to a combination therapy comprising a cell immunotherapy, such as a chimeric antigen receptor (CAR) or genetically modified T-cell receptor (TCR), in combination with a TGFβ receptor antagonist in order to prolong the induced immune response.

### BACKGROUND TO THE INVENTION

T cells of the immune system recognize and interact with specific antigens through T cell receptors (TCRs) which, upon recognition or binding with such antigens, cause activation of the cell. TCRs are expressed on the T cell surface and comprise highly variable protein chains (such as alpha (α) and beta (β) chains or gamma (γ) and delta (δ) chains), which are expressed as part of a complex with CD3 chain molecules. The CD3 chain molecules have an invariant structure and, in particular, the CD3zeta (CD3) chain is responsible for intracellular signalling upon TCR:antigen binding. The TCRs recognise antigenic peptides that are presented to it by the proteins of the major histocompatibility complex (MHC) which are expressed on the surface of all nucleated cells, e.g., antigen presenting cells and other T cell targets.

Natural Killer (NK) cells of the immune system developed earlier in evolution than T cells and bridge innate and adaptive immunity. Like T cells, NK cells recognize and interact with specific ligands on self cells, but rather than using a TCR, they rely on a plethora of activating and inhibiting signals which are integrated to result in induction or inhibition of effector functions. NK cells are innate effector cells serving as a first line of defence against certain viral infections and tumours (Biron, et al., (1999) Annu. Rev. Immunol. 17:189-220; Trinchieri (1989) Adv. Immunol. 47:187-376). Innate effector cells recognize and eliminate their targets with fast kinetics, without prior sensitization, therefore, NK cells need to sense if cells are transformed, infected, or stressed to discriminate between abnormal and healthy tissues. According to the “missing self” phenomenon (Karre, et al., (1986) Nature 319:675-678), NK cells accomplish this by looking for and eliminating cells with aberrant major histocompatibility complex (MHC) class I expression.

Cell immunotherapy is a rapidly growing field for gene therapy, which has mainly focussed on the use of genetically modified T cell receptors (TCRs) and chimeric antigen receptors (CARs) carried by T cells and/or NK cells.

Chimeric antigen receptors (CARs) have been developed as artificial immune receptors to generate novel specificities in T cells. These synthetic receptors contain a target binding domain that is associated with one or more signalling domains via a flexible linker in a single fusion molecule. The target binding domain is used to target the T cell to specific targets on the surface of pathologic cells and the signalling domains contain molecular machinery for T cell activation and proliferation. The flexible linker which passes through the T cell membrane (i.e., forming a transmembrane domain) allows for cell membrane display of the target binding domain of the CAR. CARs have successfully allowed T cells to be redirected against antigens expressed at the surface of tumour cells from various malignancies including lymphomas and solid tumours (Gross et al., (1989) Transplant Proc., 21(1 Pt 1): 127-30; Jena et al., (2010) Blood, 116(7):1035-44).

The development of CARs has comprised three generations so far. First generation CARs comprised target binding domains attached to a signalling domain derived from the cytoplasmic region of the CD3zeta or the Fc receptor gamma chains. First generation CARs were shown to successfully redirect T cells to the selected target, but they failed to provide prolonged expansion and antitumour activity in vivo. Second and third generation CARs have focussed on enhancing modified T cell survival and increasing proliferation by including co-stimulatory molecules, such as CD28, OX-40 (CD134) and 4-1BB (CD137).

Cell immunotherapy strategies have also focussed on genetically modifying TCR α- and β-chains to redirect the antigen specificity and/or optimise the antigen affinity.

T cells bearing CARs or genetically modified TCRs could be used to eliminate pathologic cells in a disease setting. One clinical aim would be to transform patient cells with recombinant DNA containing an expression construct for the CAR or TCR via a vector (e.g., a lentiviral vector) following aphaeresis and T cell or NK cell isolation. Following expansion of the T cells or NK cells, they are re-introduced into the patient with the aim of targeting and killing the pathologic target cells.

However, there is still a need in the art to improve the efficacy of cell immunotherapies. In particular, many disease cells such as malignant cancerous cells, generate an immunosuppressive microenvironment which generally down-regulates the immune response and in particular provides inhibitory signals to tumour-infiltrating lymphocytes. There is therefore a need to improve cell immunotherapies so that they can become resistant towards negative immune-modulation mediated by a hostile disease microenvironment.

### SUMMARY OF THE INVENTION

According to a first aspect of the invention, there is provided a pharmaceutical composition comprising:

(a) a TGFβ Receptor (TGFβR) antagonist which comprises a domain antibody; and

(b) a cell immunotherapy.

According to a further aspect of the invention, there is provided a polynucleotide comprising a sequence that encodes a TGFβ Receptor (TGFβR) antagonist which comprises a domain antibody, and a sequence that encodes a chimeric antigen receptor (CAR) or a T cell receptor (TCR).

According to a further aspect of the invention, there is provided an expression vector comprising the polynucleotide as defined herein.

According to a further aspect of the invention, there is provided an immunomodulatory cell comprising the polynucleotide as defined herein or the expression vector as defined herein.

According to a further aspect of the invention, there is provided an immunomodulatory cell as defined herein, for use in therapy.

According to a further aspect of the invention, there is provided a pharmaceutical composition comprising:

(1) a plurality of immunomodulatory cells as defined herein; and

(2) a pharmaceutically acceptable carrier.

According to a further aspect of the invention, there is provided a method for producing an antigen-specific immunomodulatory cell, the method comprising introducing into an immunomodulatory cell the polynucleotide or the expression vector as defined herein.

According to a further aspect of the invention, there is provided a method of treatment comprising administering the expression vector or the immunomodulatory cell as defined herein, to a subject.

According to a further aspect of the invention, there is provided a method of treatment comprising administering to a subject:

(a) a cell immunotherapy, and

(b) a TGFβ Receptor (TGFβR) antagonist which comprises a domain antibody.

According to a further aspect of the invention, there is provided a kit for the treatment of a disease selected from: cancer, an autoimmune disease or infection, comprising the pharmaceutical composition, the polynucleotide, the expression vector, or the immunomodulatory cell as defined herein.

### BRIEF DESCRIPTION OF THE FIGURES

FIG. 1 depicts results of Meso Scale Discovery (MSD) based ligand binding assays to quantify the expression of interferon gamma (IFN-γ) in human CD4⁺ and CD8⁺ T-lymphocytes at various conditions. T-lymphocytes activated with anti-CD3 and anti-CD28 antibodies, were incubated with varying concentrations of TGFβR antagonist dAb comprising the amino acid sequence of SEQ ID NO: 72, and/or human TGFβ for 24, 48, and 72 hour time points.

FIG. 2 depicts results of MSD based ligand binding assays to quantify the expression of IL-2 in human CD4⁺ and CD8⁺ T-lymphocytes at various conditions. T-lymphocytes activated with anti-CD3 and anti-CD28 antibodies, were incubated with varying concentrations of TGFβR antagonist dAb comprising the amino acid sequence of SEQ ID NO: 72, and/or human TGFβ for 24, 48, and 72 hour time points.

FIG. 3 depicts results of MSD based ligand binding assays to quantify the expression of IL-6 in human CD4⁺ and CD8⁺ T-lymphocytes at various conditions. T-lymphocytes activated with anti-CD3 and anti-CD28 antibodies, were incubated with varying concentrations of TGFβR antagonist dAb comprising the amino acid sequence of SEQ ID NO: 72, and/or human TGFβ for 24, 48, and 72 hour time points.

FIG. 4 depicts results of MSD based ligand binding assays to quantify the expression of IL-10 in human CD4⁺ and CD8⁺ T-lymphocytes at various conditions. T-lymphocytes activated with anti-CD3 and anti-CD28 antibodies, were incubated with varying concentrations of TGFβR antagonist dAb comprising the amino acid sequence of SEQ ID NO: 72, and/or human TGFβ for 24, 48, and 72 hour time points.

FIG. 5 depicts results of MSD based ligand binding assays to quantify the expression of IL-17 in human CD4⁺ and CD8⁺ T-lymphocytes at various conditions. T-lymphocytes activated with anti-CD3 and anti-CD28 antibodies, were incubated with varying concentrations of TGFβR antagonist dAb comprising the amino acid sequence of SEQ ID NO: 72, and/or human TGFβ for 24, 48, and 72 hour time points.

FIG. 6 depicts results of flow cytometry assays to quantify cell surface expression of CD103 in human CD4⁺ and CD8⁺ T-lymphocytes at various conditions. T-lymphocytes activated with anti-CD3 and anti-CD28 antibodies, were incubated with varying concentrations of TGFβR antagonist dAb comprising the amino acid sequence of SEQ ID NO: 72, and/or human TGFβ for 24, 48, and 72 hour time points.

FIG. 7 depicts results of flow cytometry assays to quantify cell surface expression of CXCR4 in human CD4⁺ and CD8⁺ T-lymphocytes at various conditions. T-lymphocytes activated with anti-CD3 and anti-CD28 antibodies, were incubated with varying concentrations of TGFβR antagonist dAb comprising the amino acid sequence of SEQ ID NO: 72, and/or human TGFβ for 24, 48, and 72 hour time points.

FIG. 8 depicts results of flow cytometry assays to quantify cell surface expression of OX40 in human CD4⁺ and CD8⁺ T-lymphocytes at various conditions. T-lymphocytes activated with anti-CD3 and anti-CD28 antibodies, were incubated with varying concentrations of TGFβR antagonist dAb comprising the amino acid sequence of SEQ ID NO: 72, and/or human TGFβ for 24, 48, and 72 hour time points.

FIG. 9 depicts results of flow cytometry assays to quantify cell surface expression of PD1 in human CD4⁺ and CD8⁺ T-lymphocytes at various conditions. T-lymphocytes activated with anti-CD3 and anti-CD28 antibodies, were incubated with varying concentrations of TGFβR antagonist dAb comprising the amino acid sequence of SEQ ID NO: 72, and/or human TGFβ for 24, 48, and 72 hour time points.

### DETAILED DESCRIPTION OF THE INVENTION

### Definitions

Unless defined otherwise, all technical and scientific terms used herein have the same meaning as is commonly understood by one of skill in the art (e.g., in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 2^{nd} ed. (1989) Cold Spring Harbor Laboratory Press, Cold Spring Harbor, N.Y. and Ausubel et al., Short Protocols in Molecular Biology (1999) 4th Ed, John Wiley & Sons, Inc., which are incorporated herein by reference in their entirety) and chemical methods. All patents and publications referred to herein are incorporated by reference in their entirety.

The term “comprising” encompasses “including” or “consisting” e.g. a composition “comprising” X may consist exclusively of X or may include something additional, e.g., X+Y.

The term “consisting essentially of” limits the scope of the feature to the specified materials or steps and those that do not materially affect the basic characteristic(s) of the claimed feature.

The term “consisting of” excludes the presence of any additional component(s).

The term “about” as used herein when referring to a measurable value such as an amount, a temporal duration, and the like, is meant to encompass variations of ±20% or ±10%, including ±5%, ±1%, and ±0.1% from the specified value.

The term “cell immunotherapy” as used herein, refers to a type of therapy in which immunomodulatory cells are genetically modified in order to target disease and then introduced into the patient. Areas of key focus are introducing chimeric antigen receptors (CARs) or genetically modified T cell receptors (TCRs) onto immunomodulatory cells in order to make them target specific.

The terms “adoptive cellular therapy” or “adoptive immunotherapy” as used herein, refer to the adoptive transfer of human T lymphocytes or NK lymphocytes that are engineered by gene transfer to express CARs or genetically modified TCRs, specific for surface antigens or peptide MHC complexes expressed on target cells. This can be used to treat a range of diseases depending upon the target chosen, e.g., tumour specific antigens to treat cancer. Adoptive cellular therapy involves removing a portion of a donor's or the patient's white blood cells using a process called leukapheresis. The T cells or NK cells may then be expanded and mixed with expression vectors comprising the CAR/TCR polynucleotide in order to transfer the CAR/TCR scaffold to the T cells or NK cells. The T cells or NK cells are expanded again and at the end of the expansion, the engineered T cells or NK cells are washed, concentrated, and then frozen to allow time for testing, shipping and storage until a patient is ready to receive the infusion of engineered cells.

The term “autologous” as used herein, refers to cells from the same subject. The term “allogeneic” as used herein, refers to cells of the same species that differ genetically to the cell in comparison.

The term “immunomodulatory cell” as used herein, refers to a cell that functions in an immune response, or a progenitor or progeny thereof. Examples of immunomodulatory cells include: T cells (also known as T-lymphocytes) which may be inflammatory, cytotoxic, regulatory or helper T cells; B cells (or B-lymphocytes) which may be plasma or memory B-cells; natural killer cells; neutrophils; eosinophils; basophils; mast cells; dendritic cells; or macrophages.

The term “T cell receptor” (“TCR”) as used herein, refers to the receptor present on the surface of T cells which recognises fragments of antigen as peptides bound to major histocompatibility complex (MHC) molecules. Native TCRs exist in αβ and γδ forms, which are structurally similar but exist in different locations and are thought to have different functions. The extracellular portion of the TCR has two constant domains and two variable domains. The variable domains contain polymorphic loops which form the binding site of the TCR and are analogous to complementarity determining regions (CDRs) in antibodies. In the context of cell immunotherapies, the TCR is usually genetically modified to change or improve its antigen recognition. For example, WO01/055366 and WO2006/000830, which are herein incorporated by reference, describe retrovirus-based methods for transfecting T cells with heterologous TCRs.

The term “chimeric antigen receptors” (“CARs”) as used herein, refers to an engineered receptor which consists of an extracellular target binding domain (which is usually derived from a monoclonal antibody), a spacer region, a transmembrane region, and one or more intracellular effector domains. CARs have also been referred to as chimeric T cell receptors or chimeric immunoreceptors (CIRs). CARs are genetically introduced into hematopoietic cells, such as T cells, to redirect specificity for a desired cell-surface antigen or MHC-peptide complex.

The term “target binding domain” as used herein is defined as an oligo- or polypeptide that is capable of binding a specific target, such as an antigen or ligand. In particular, the target may be a cell surface molecule. For example, the target binding domain may be chosen to recognise a target that acts as a cell surface marker on pathogenic cells, including pathogenic human cells, associated with a particular disease state.

The term “spacer region” as used herein, refers to an oligo- or polypeptide that functions to link the transmembrane domain to the target binding domain. This region may also be referred to as a “hinge region” or “stalk region”.

The term “domain” refers to a folded protein structure which retains its tertiary structure independent of the rest of the protein. Generally domains are responsible for discrete functional properties of proteins and in many cases may be added, removed or transferred to other proteins without loss of function of the remainder of the protein and/or of the domain.

The term “transmembrane domain” as used herein refers to the part of the CAR molecule which traverses the cell membrane.

The term “intracellular effector domain” (also referred to as the “signalling domain”) as used herein refers to the domain in the CAR which is responsible for intracellular signalling following the binding of the target binding domain to the target. The intracellular effector domain is responsible for the activation of at least one of the normal effector functions of the immune cell in which the CAR is expressed. For example, the effector function of a T cell can be a cytolytic activity or helper activity including the secretion of cytokines. Alternatively, the intracellular effector domain may be directed to another type of immunomodulatory cell such as an NK cell and may comprise NKG2D, DAP10, or DAP12 or a functional fragment and/or derivative thereof.

The term “TGFβR antagonist” refers to an antagonist (also referred to as an antigen binding protein herein) which is able to prevent the TGFβ receptor (TGFβR) from signalling. A person skilled in the art would understand that this can be achieved by either binding the cytokine (i.e., TGFβ) which activates the signalling of TGFβR, or the receptor itself. Therefore this term encompasses both antagonists which bind TGFβ and antagonists which bind TGFβR. In one embodiment, the antagonist of the disclosure can neutralize TGFβ signalling through TGFβRII. By “neutralizing”, it is meant that the normal signalling effect of TGFβ is blocked such that the presence of TGFβ has a neutral effect on TGFβRII signalling.

The term “domain antibody” as used herein refers to a folded polypeptide domain comprising sequences characteristic of antibody variable domains. It therefore includes complete antibody variable domains such as V_{H}, V_{HH} and V_{L} and modified antibody variable domains, for example, in which one or more loops have been replaced by sequences which are not characteristic of antibody variable domains, or antibody variable domains which have been truncated or comprise N- or C-terminal extensions, as well as folded fragments of variable domains which retain at least the binding activity and specificity of the full-length domain. A domain antibody is capable of binding an antigen or epitope independently of a different variable region or domain. A “domain antibody” or “dAb™” may also be referred to as a “single variable domain”. A domain antibody may be a human domain antibody, but also includes single domain antibodies from other species such as rodent (for example, as disclosed in WO 00/29004), nurse shark and Camelid V_{HH} dAbs. Camelid V_{HH} are immunoglobulin single variable domain polypeptides that are derived from species including camel, llama, alpaca, dromedary, and guanaco, which produce heavy chain antibodies naturally devoid of light chains. Such V_{HH} domains may be humanised according to standard techniques available in the art, and such domains are considered to be “domain antibodies”. As used herein V_{H} includes camelid V_{HH} domains. NARV are another type of immunoglobulin domain antibody, which were identified in cartilaginous fish including the nurse shark (Shao et al., (2006) Mol. Immunol. 44, 656-665). These domains are also known as Novel Antigen Receptor variable region (commonly abbreviated to V(NAR) or NARV).

The term “dual domain antibody”, “dual dAb” or “dual specific ligand” as used herein refers to a molecule which comprises two domain antibodies connected directly or via a linker. In one embodiment, the dual domain antibody comprises a first domain antibody and a second domain antibody, wherein the binding sites or variable domains are capable of binding to two antigens (e.g., different antigens or two copies of the same antigen) or two epitopes on the same antigen which are not normally bound by a monospecific immunoglobulin. For example, the two epitopes may be on the same antigen, but are not the same epitope or sufficiently adjacent to be bound by a monospecific ligand. In one embodiment, dual domain antibody comprises two domain antibodies which have different specificities, and do not contain mutually complementary variable domain pairs (i.e., V_{H}/V_{L} pairs) which have the same specificity (i.e., do not form a unitary binding site). Details of dual specific ligands are found in WO2003/002609, WO2004/003019, WO2008/096158, WO2004/058821 and WO2013/014208, which are herein incorporated by reference.

In one embodiment, the antagonist comprises a dimer of the dual domain antibodies disclosed herein. As used herein, the term “dimer” means a polypeptide complex which comprises two antigen binding constructs, i.e., two chains that associate with one another to form a dimer. A dimer may be a homodimer, comprising two identical antigen binding constructs of the invention or a heterodimer comprising two different antigen binding constructs of the invention. Homodimers and heterodimers of the present invention may have improved properties, such as affinity, for the target molecule.

Multispecific domain antibody (dAb) multimers are also provided. This includes a dAb multimer comprising an anti-TGFβRII immunoglobulin domain antibody and one or more domain antibodies, each of which binds to a different target (e.g. a target other than TGFβRII). In one embodiment, a bispecific dAb multimer is provided, e.g., a dab multimer comprising one or more anti-TGFβRII domain antibodies and one or more domain antibodies which bind to a second, different target. In an embodiment a trispecific dAb multimer is provided.

Antagonists according to the disclosure, including dAb monomers, dimers and trimers, can be linked to an antibody Fc region, comprising one or both of C_{H}2 and C_{H}3 domains, and optionally a hinge region. For example, vectors encoding ligands linked as a single nucleotide sequence to an Fc region may be used to prepare such polypeptides. In an embodiment there is provided a dAb-Fc fusion which comprises a domain antibody attached to a single chain Fc region of an antibody.

The term “single chain Fc region of an antibody” or “antibody Fc region” as used herein refers to a single heavy chain Fc region of an IgG, such as an IgG1, IgG2, IgG3, IgG4 or IgG4PE, or an IgA antibody. A single heavy chain Fc region may comprise one or more of the C_{H}2 and C_{H}3 constant region antibody domains. In addition to comprising the C_{H}2 and/or C_{H}3 constant region antibody domains, the single heavy chain Fc region of an antibody may further comprise a hinge region of an antibody (such a region normally found between the C_{H}1 and C_{H}2 domains). In one embodiment, the single chain Fc region of an antibody is a single IgG1 heavy chain, for example a single IgG1 heavy chain comprising the C_{H}2 and C_{H}3 antibody constant domains.

The Fc region of an antibody may be selected for its degree of effector function. The term “effector function” as used herein is meant to refer to one or more of Antibody Dependent Cell-mediated Cytotoxicity (ADCC), Complement-Dependent Cytotoxicity (CDC) mediated responses, Fc-mediated phagocytosis and antibody recycling via the FcRn receptor. For IgG antibodies, effector functionalities including ADCC and CDC are mediated by the interaction of the heavy chain constant region with a family of Fcγ receptors present on the surface of immune cells. In humans these include FcγRI (CD64), FcγRII (CD32) and FcγRIII (CD16). Interaction between the antigen binding protein bound to the antigen and the formation of the Fc/Fcγ complex induces a range of effects including cytotoxicity, immune cell activation, phagocytosis and release of inflammatory cytokines.

The interaction between the constant region of an antigen binding protein and various Fc receptors (FcR) is believed to mediate the effector functions of the antigen binding protein. Significant biological effects can be a consequence of effector functionality, in particular, antibody-dependent cellular cytotoxicity (ADCC), fixation of complement (complement dependent cytotoxicity or CDC), and half-life/clearance of the antigen binding protein. Usually, the ability to mediate effector function requires binding of the antigen binding protein to an antigen and not all antigen binding proteins will mediate every effector function.

Effector function can be measured in a number of ways including for example via binding of the FcγRIII to Natural Killer cells or via FcγRI to monocytes/macrophages to measure for ADCC effector function. For example an antigen binding protein of the present invention can be assessed for ADCC effector function in a Natural Killer cell assay. Examples of such assays can be found in Shields et al., (2001) J. Biol. Chem., 276: 6591-6604; Chappel et al., (1993) J. Biol. Chem., 268: 25124-25131; Lazar et al., (2006) PNAS USA, 103: 4005-4010. Examples of assays to determine CDC function include that described in Patel & Boyd (1995) J. Immunol. Methods, 184: 29-38.

Some isotypes of human constant regions, in particular IgG4 and IgG2 isotypes, essentially lack the functions of (a) activation of complement by the classical pathway; and (b) antibody-dependent cellular cytotoxicity. Various modifications to the heavy chain constant region of antigen binding proteins may be carried out depending on the desired effector property. IgG1 constant regions containing specific mutations have separately been described to reduce binding to Fc receptors and therefore reduce ADCC and CDC (Duncan et al., (1988) Nature, 332: 563-564; Lund et al., (1991) J. Immunol. 147: 2657-2662; Chappel et al., (1991) PNAS USA 88: 9036-9040; Burton and Woof, (1992) Adv. Immunol. 51: 1-84; Morgan et al., (1995) Immunology 86: 319-324; Hezareh et al., (2001) J. Virol. 75 (24): 12161-12168).

Human IgG1 constant regions containing specific mutations or altered glycosylation, e.g., on residue Asn297, have also been described to enhance binding to Fc receptors. In some cases these mutations have also been shown to enhance ADCC and CDC (Lazar et al., (2006) PNAS USA 103: 4005-4010; Shields et al., (2001) J. Biol. Chem. 276: 6591-6604; Nechansky et al., (2007) Mol. Immunol. 44: 1815-1817).

Naturally occurring autoantibodies exist in humans that can bind to proteins. Autoantibodies can thus bind to endogenous proteins (present in naïve subjects) as well as to proteins or peptides which are administered to a subject for treatment. Therapeutic protein-binding autoantibodies and antibodies that are newly formed in response to drug treatment are collectively termed anti-drug antibodies (ADAs). Pre-existing antibodies against molecules such as therapeutic proteins and peptides, administered to a subject can affect their efficacy and could result in administration reactions, hypersensitivity, altered clinical response in treated patients and altered bioavailability by sustaining, eliminating or neutralizing the molecule. It could be advantageous to provide molecules for therapy which comprise human immunoglobulin (antibody) single variable domains or dAbs™ which have reduced immunogenicity (i.e., reduced ability to bind to pre-existing ADAs when administered to a subject, in particular a human subject).

Thus in one embodiment of the present invention there is provided a modified domain antibody which has reduced ability to bind to pre-existing antibodies (ADAs) as compared to the equivalent unmodified molecule. By reduced ability to bind it is meant that the modified molecule binds with a reduced affinity or reduced avidity to a pre-existing ADA. Said modified domain antibodies comprise one or more modifications selected from: (a) a C-terminal addition, extension, deletion or tag, and/or (b) one or more amino acid framework substitutions.

In one embodiment the modified domain antibody comprises:

a) a C-terminal sequence consisting of the sequence VTVS(S)ₙX [for a VH dAb™] or VEIKₚR_{q}X [for a VL dAb™]; and also optionally

b) one or more amino acid substitutions at positions 14, 41, 108, 110, or 112 compared to a human germline framework sequence

wherein:

n represents an integer independently selected from 0 or 1;

p and q each represent 0 or 1 such that when p represents 1 q may be 0 or 1 and such that when p represents 0, q also represents 0;

X may be present or absent, and if present represents an amino acid extension of 1 to 8 amino acids residues;

with the further proviso that if X is absent;

for a VH dAb™: n is 0 and/or the dAb™ ending in VTVS(S)n [for a VH dAb™] comprises one or more of said amino acid substitutions;

for a VL dAb™: p and/or q is 0, and/or the dAb™ ending in VEIKₚR_{q}X comprises one or more of said amino acid substitutions.

Immunoassays well known to those skilled in the art can be used to confirm that the modified dAbs have the desired reduced binding to ADAs.

In a further aspect of this embodiment, said modified domain antibody with reduced binding to pre-existing ADAs has one or more amino acid substitutions wherein said one or more amino acid substitutions are selected from the group consisting of a P14A substitution, a P41A substitution, a L108A substitution, a T110A substitution, a S112A substitution, a P14K substitution, a P14Q substitution, and a P14T substitution.

In a further aspect of this embodiment, X is present, and is an extension of 1 to 8 amino acids, in particular an extension of 1 to 8 amino acids which comprises an alanine residue, for example a single alanine extension, or an AS, AST, ASTK, ASTKG, ASTKGP extension.

In one embodiment the modified domain antibody can comprise a tag present at the C terminus. The tag can be any tag known in the art for example affinity tags such as myc-tags, FLAG tags, his-tags, chemical modification such as PEG, or protein domains such as the antibody Fc domain. The C terminal addition or extension or tag can be present as a direct fusion or conjugate with the C terminus of the molecule.

“Affinity” is the strength of binding of one molecule, e.g., the target binding protein of the CAR molecule of the invention, to another, e.g. its target antigen, at a single binding site. The binding affinity of an antigen binding protein to its target may be determined by equilibrium methods (e.g., enzyme-linked immunoabsorbent assay (ELISA) or radioimmunoassay (RIA)), or kinetics (e.g. BIACORE™ analysis).

“Avidity” is the sum total of the strength of binding of two molecules to one another at multiple sites, e.g. taking into account the valency of the interaction.

The phrases, “half-life” (“t_{1/2}”) and “serum half-life”, refer to the time taken for the serum (or plasma) concentration of an antigen binding protein in accordance with the disclosure to reduce by 50%, in vivo, for example due to degradation of the antigen binding protein and/or clearance or sequestration of the antigen binding protein by natural mechanisms.

The term “epitope” as used herein refers to that portion of the antigen that makes contact with a particular binding domain, e.g., the antigen binding protein or the target binding domain of the CAR molecule. The epitope may comprise a particular sequence or confirmation of amino acid residues or a particular confirmation of other molecular structures, such as sugar residues (e.g., a specific pattern of sulphated sugar residues). An epitope may be linear or conformational/discontinuous. A conformational or discontinuous epitope may comprise amino acid residues that are separated by other sequences, i.e., not in a continuous sequence in the antigen's primary sequence. Although the residues may be from different regions of the peptide chain, they are in close proximity in the three dimensional structure of the antigen. In the case of multimeric antigens, a conformational or discontinuous epitope may include residues from different peptide chains. Particular residues comprised within an epitope can be determined through computer modelling programs or via three-dimensional structures obtained through methods known in the art, such as X-ray crystallography.

The term “pathogen” refers to a virus, bacteria, fungi, parasite or protozoa which is capable of causing disease.

The term “cancer” (sometimes also referred to as “neoplasia”) refers to a disease caused by an uncontrolled division of abnormal cells in a part of the body. The uncontrolled division can often result in a mass, commonly referred to as a “tumour” or “neoplasm”.

The term “tumour associated antigen” or “tumour antigen” as used herein, refers to an antigen expressed on a tumour cell. This antigen may be uniquely or differentially expressed on a tumour cell when compared to a normal, i.e., non-cancerous, cell.

The term “pharmaceutical composition” refers to a composition formulated in pharmaceutically-acceptable or physiologically-acceptable solutions for administration to a cell or animal. The compositions of the invention may be administered in combination with other agents as well, provided that the additional agents do not adversely affect the ability of the composition to deliver the intended therapy.

The term “vector” or “nucleic acid vector” refers to a vehicle which is able to artificially carry foreign (i.e., exogenous) genetic material into another cell, where it can be replicated and/or expressed. Examples of vectors include non-mammalian nucleic acid vectors, such as plasmids, bacterial artificial chromosomes (BACs), yeast artificial chromosomes (YACs), P1-derived artificial chromosomes (PACs), cosmids or fosmids.

Other examples of vectors include viral vectors, such as retroviral, lentiviral and adeno-associated viral (AAV) vectors, which are of particular interest in the present application. Lentiviral vectors, such as those based upon Human Immunodeficiency Virus Type 1 (HIV-1) are widely used as they are able to integrate into non-proliferating cells. Viral vectors can be made replication defective by splitting the viral genome into separate parts, e.g., by placing on separate plasmids. For example, the so-called first generation of lentiviral vectors, developed by the Salk Institute for Biological Studies, was built as a three-plasmid expression system consisting of a packaging expression cassette, the envelope expression cassette and the vector expression cassette. The “packaging plasmid” contains the entire gagpol sequences, the regulatory (tat and rev) and the accessory (vif, vpr, vpu, net) sequences. The “envelope plasmid” holds the Vesicular stomatitis virus glycoprotein (VSVg) in substitution for the native HIV-1 envelope protein, under the control of a cytomegalovirus (CMV) promoter. The third plasmid (the “transfer plasmid”) carries the Long Terminal Repeats (LTRs), encapsulation sequence (ψ), the Rev Response Element (RRE) sequence and the CMV promoter to express the transgene inside the host cell.

The second lentiviral vector generation was characterized by the deletion of the virulence sequences vpr, vpu and nef. The packaging vector was reduced to gag, pol, tat and rev genes, therefore increasing the safety of the system.

To improve the lentiviral system, the third-generation vectors have been designed by removing the tat gene from the packaging construct and inactivating the LTR from the vector cassette, therefore reducing problems related to insertional mutagenesis effects.

The various lentivirus generations are described in the following references: First generation: Naldini et al., (1996) Science 272(5259): 263-7; Second generation: Zufferey et al., (1997) Nat. BlotechnoZ 15(9): 871-5; Third generation: Dull et aZ, (1998) J. Virot. 72(11): 8463-7, all of which are incorporated herein by reference in their entirety. A review on the development of lentiviral vectors can be found in Sakuma et al., (2012) Biochem. J. 443(3): 603-18 and Picanco-Castro et al., (2008) Exp. Opin. Therap. Patents 18(5):525-539.

The term “promoter” refers to a sequence that drives gene expression. They may be inducible (i.e., require an external trigger in order to drive expression) or constitutive (i.e., they are constantly active and therefore continuously driving expression). In order to drive a high level of expression, it may be beneficial to use a high efficiency promoter, such as a non-retroviral, high efficiency promoter. Examples of suitable promoters may include a promoter such as the human cytomegalovirus (CMV) immediate early promoter, spleen focus-forming virus (SFFV) promoter, Rous sarcoma virus (RSV) promoter, human phosphoglycerate kinase (hPGK) promoter or human elongation factor 1-alpha (pEF) promoter.

The term “operably linked” refers to when the components are arranged so as to permit them to function in their intended manner. For example, it refers to the functional linkage between a promoter and a further polynucleotide sequence (e.g., a polynucleotide of interest) wherein the promoter directs transcription of the further polynucleotide sequence.

The term “expression cassette” refers to the part of a vector which can express RNA, and subsequently a protein. The cassette often contains the gene of interest. In one embodiment, the expression cassette has its 3′ and 5′ ends adapted for insertion into a vector, e.g., it has restriction enzyme sites at each end. The cassette can be removed and inserted into a plasmid or a viral vector as a single unit.

The terms “individual”, “subject” and “patient” are used herein interchangeably. In one embodiment, the subject is a mammal, such as a mouse, a primate, for example a marmoset or monkey, or a human. In a further embodiment, the subject is a human.

The invention described herein may also be used in methods of treatment of a subject in need thereof. Treatment can be therapeutic, prophylactic or preventative. Treatment encompasses alleviation, reduction, or prevention of at least one aspect or symptom of a disease and encompasses prevention or cure of the diseases described herein.

The invention described herein is used in an “effective amount” for therapeutic, prophylactic or preventative treatment. A therapeutically effective amount of the cell immunotherapy and/or TGFβR antagonist described herein is an amount effective to ameliorate or reduce one or more symptoms of, or to prevent or cure, the disease.

### Pharmaceutical Compositions

According to a first aspect of the invention, there is provided a pharmaceutical composition comprising:

(a) TGFβ Receptor (TGFβR) antagonist which comprises a domain antibody; and

(b) a cell immunotherapy.

Many disease cells, such as malignant cancerous cells, generate an immunosuppressive microenvironment which down-regulates the immune response. In order to improve the efficacy of cell immunotherapies, the present inventors have found that the addition of a TGFβ Receptor (TGFβR) antagonist to the cell immunotherapy treatment helps to prolong the duration of the immune response. Without being bound by theory, this is thought to be due to the neutralisation of TGFβ signalling which is understood to have an immunosuppressive role in the tumour microenvironment. Overexpression of TGFβ in advanced tumours has been associated with metastasis and poor prognosis and is thought to be due partly to the role of TGFβ in converting effector T cells, which normally attack cancer with an inflammatory (immune) reaction, into regulatory (suppressor) T cells, which turn off the inflammatory reaction, i.e., resulting in immunosuppression. TGFβ signalling also inhibits the activation and function of NK cells, which are important in tumour immunosurveillance.

Furthermore, the use of a TGFβR antagonist which comprises a domain antibody provides several advantages. The optimal size of the antagonist used needs to be balanced to ensure it has sufficient transduction within the disease site to have an effect, while not travelling so far from the disease site to cause unwanted side effects. The small size of domain antibodies (or small binding proteins comprising such domain antibodies) means that they have a short in vivo half-life, especially when compared to larger antigen binding proteins such as monoclonal antibodies (mAbs), which helps to reduce any systemic side effects of the antagonist. Domain antibodies also have a higher volume of distribution than monoclonal antibodies which is thought to help facilitate penetration of solid tumour masses and particularly useful for overcoming the immunosuppressive tumour microenvironment. Therefore, the present invention is particularly well suited for treating large tumours which have an established immunosuppressive microenvironment.

Using smaller antagonists also has production advantages. For example, in order to minimise the cost of goods and number of transfection steps required, it would be advantageous to have the CAR/TCR and TGFβR antagonist encoded within the same expression vector. However vectors are size limited, and it is therefore helpful to have a small TGFβR antagonist to ensure that it can be encoded within the same vector as the large CAR/TCR molecule.

Compositions of the invention include pharmaceutical compositions comprising genetically modified immunomodulatory cells or their progenitors and a TGFβ Receptor (TGFβR) antagonist which comprises a domain antibody. Administration can be autologous or heterologous (i.e., allogeneic). For example, immunomodulatory cells, or progenitors can be obtained from one subject, and administered to the same subject (i.e., autologous) or a different, compatible subject (i.e., allogeneic).

Examples of additional pharmaceutical composition ingredients include, without limitation, any adjuvants, carriers, excipients, glidants, sweetening agents, diluents, preservatives, dyes/colourants, flavour enhancers, surfactants, wetting agents, dispersing agents, suspending agents, stabilizers, isotonic agents, solvents, surfactants, emulsifiers, buffers (such as phosphate buffered saline (PBS)), carbohydrates (such as glucose, mannose, sucrose or dextrans), amino acids, antioxidants or chelating agents (such as EDTA or glutathione).

In one embodiment, the pharmaceutical composition additionally comprises a pharmaceutically acceptable excipient, carrier, or diluent. The carrier, excipient or diluent must be “acceptable” in the sense of being compatible with the other ingredients of the composition and not deleterious to the recipient thereof. According to the present invention any excipient, vehicle, diluents or additive used would have to be compatible with the cell immunotherapy and TGFβR antagonist. Standard texts known in the art, such as “Remington's Pharmaceutical Science”, 17th Edition, 1985, incorporated herein by reference, may be consulted to prepare suitable preparations.

Pharmaceutical compositions may be administered by injection or continuous infusion (examples include, but are not limited to, intravenous, intratumoural, intraperitoneal, intradermal, subcutaneous, intramuscular and intraportal). In one embodiment, the composition is suitable for intravenous administration. When administering a therapeutic composition of the present invention (e.g., a pharmaceutical composition containing a genetically modified immunoresponsive cell and TGFβR antagonist which comprises a domain antibody), it will generally be formulated in a unit dosage injectable form (solution, suspension, emulsion). Pharmaceutical compositions may be suitable for topical administration (which includes, but is not limited to, epicutaneous, inhaled, intranasal or ocular administration) or enteral administration (which includes, but is not limited to, oral or rectal administration).

Methods for the preparation of such pharmaceutical compositions are well known to those skilled in the art. Other excipients may be added to the composition as appropriate for the mode of administration and the particular protein used.

Effective doses and treatment regimes for administering the composition of the present invention may be dependent on factors such as the age, weight and health status of the patient and disease to be treated. Such factors are within the purview of the attending physician.

### Transforming Growth Factor Beta Receptor Antagonists

Transforming Growth Factor β (TGF beta; TGFβ) is a signalling molecule that mediates signal transduction into cells through binding to a TGFβ receptor (TGF beta Receptor; TGFβR). TGFβ signalling activity regulates cell differentiation and growth, the nature of its effect, i.e., as cell growth-promoter, growth-suppressor or inducer of other cell functions, being dependent on cell type (see Roberts, et al., The transforming growth factor-betas, Peptide Growth Factors and Their Receptors, Part I, ed. by Sporn, M. B. & Roberts, A. B., Springer-Verlag, Berlin, (1990) 419-472).

TGFβ is produced by a wide variety of cell types, and its cognate receptors are expressed in a wide variety of organs and cells (see Shi and Massague (2003) Cell, 113(6): 685-700). TGFβ receptors have been identified to fall into three types: TGFβRI (TGF beta type I receptor; see Franzen et al., (1993) Cell, 75(4): 681; and GenBank Accession No: L11695); TGFβRII (TGF beta type II receptor; see Herbert et al., (1992) Cell, 68(4): 775; GenBank Accession No: M85079) and TGFβRIII (TGF beta type III receptor; see Lopez-Casillas (1991) Cell, 67(4): 785; Gen Bank Accession No: L07594).

TGFβ signalling is mediated through its binding to both TGFβRI and RII. When the ligand binds to the extracellular ligand binding domain, the two receptors are brought together, allowing RII to phosphorylate RI and begin the signalling cascade through the phosphorylation of Smad proteins (see Shi and Massague as referred to above).

Three isoforms of TGFβ have been identified in mammals: TGFβ1, TGFβ2, and TGFβ3. Each isoform is multifunctional and acts in self-regulatory feedback mechanisms to control bioavailability for developmental processes and to maintain tissue homeostasis (as reviewed in ten Dijke and Arthur (2007) Nat. Rev. Mol. Cell Biol., 8: 857-869).

In one embodiment, the TGFβR antagonist binds to TGFβR or TGFβ, i.e., the TGFβR antagonist is an anti-TGFβR antigen binding protein, or an anti-TGFβ antigen binding protein. In a further embodiment, the TGFβR antagonist binds to TGFβR.

In one embodiment, the TGFβR antagonist binds to TGFβ type I receptor (TGFβRI), TGFβ type II receptor (TGFβRII) or TGFβ type III receptor (TGFβRIII). In a further embodiment, the TGFβR antagonist binds to TGFβ type II receptor (TGFβRII).

In an alternative embodiment, the TGFβR antagonist binds to TGFβ. In a further embodiment, the TGFβR antagonist binds to TGFβ1, TGFβ2 or TGFβ3.

The antagonists of the present invention require the presence of at least one domain antibody. It will be understood that such antagonists do not include monoclonal antibodies because domain antibodies are capable of binding an antigen or epitope independently of a different variable region or domain.

In one embodiment, the TGFβR antagonist is selected from: a domain antibody (also known as a single variable domain or a dAb), a dual domain antibody (i.e., a dual dAb), or a domain antibody attached to a single chain Fc region of an antibody (i.e., a dAb-Fc). The optimal size of the antagonist needs to have a sufficient effect on the disease site without causing unwanted systemic side effects. The choice of the size of the molecule may depend upon if an effect is only required in the immediate environment (i.e., the environment closely surrounding the site of administration or secretion), or alternatively, if a more penetrative effect is required.

In a further embodiment, the TGFβR antagonist is a domain antibody. The use of domain antibodies in the context of the present invention has multiple advantages as detailed herein, in particular due to their small size. Furthermore, in the context of the present invention, the antagonist is only required to bind to a molecule expressed on the T cell surface (e.g., the TGFβ receptor) or an interacting molecule nearby (e.g., TGFβ in the tumour microenvironment), therefore a domain antibody is an ideal size for the antagonist because it will have an effect on the immediate environment without causing any systemic effects.

In one embodiment, the TGFβR antagonist is a domain antibody attached to a single chain Fc region of an antibody (i.e., a dAb-Fc).

In one embodiment, the domain antibody attached to the N-terminus end of the Fc region of an antibody, is a heavy or light chain domain antibody wherein the light chain domain antibody may be a kappa or lambda light chain.

In one embodiment, the domain antibody attached to the C-terminus end of the Fc region of an antibody, is a heavy or light chain domain antibody wherein the light chain domain antibody may be a kappa or lambda light chain.

In one embodiment, the TGFβR antagonist is a dual domain antibody. Each domain antibody in the dual domain antibody may be the same or different, and such domain antibodies may bind the same epitope on the target or different epitopes. In one embodiment, the TGFβR antagonist is a dual domain antibody comprising two anti-TGFβR dAbs. In an alternative embodiment, the TGFβR antagonist is a dual domain antibody comprising an anti-TGFβR dAb (a first dAb) that binds to TGFβRII and a second dAb that binds serum albumin (SA), the second dAb binding SA.

In a further embodiment, the dual domain antibody comprises two domain antibodies separated by a single chain Fc region of an antibody wherein each domain antibody is capable of binding to the target (e.g., a dAb-Fc-dAb). By separated it is meant that the domain antibodies are not directly attached to one another. In one aspect the domain antibodies are located at opposite ends of the Fc region. One domain antibody is attached to the N-terminus and the other is attached to the C-terminus.

It will be understood that in any of the embodiments described herein, the domain antibody may be a light chain domain antibody or heavy chain domain antibody.

Domain antibodies can be attached directly to the Fc region of an antibody or indirectly through a linker. In constructs where the N-terminus of a domain antibody is fused to the C-terminus of a Fc region of an antibody, a peptide linker may enhance antigen binding of the domain antibody. Indeed, the N-terminal end of a domain antibody is located closely to the complementarity-determining regions (CDRs) involved in antigen-binding activity. Thus a peptide linker may act as a spacer between the epitope-binding, and the constant domain of the protein scaffold, which may allow the domain antibody CDRs to more easily reach the antigen, and in some circumstances bind with higher affinity. Furthermore, certain peptide linkers, for examples those greater than 7 amino acids in length, may promote and enable the association of a heavy chain domain antibody attached to the N-terminus of the Fc region of an antibody to a light chain domain antibody attached to the C-terminus of the Fc region of an antibody, in heterodimers and homodimers as described herein. Such association may enhance antigen binding and/or other properties of the antagonists of the present invention.

When fused at the C-terminal end of the Fc region of the antibody, each domain antibody may be located in the vicinity of the C_{H}3 domains of the Fc portion. This is not expected to impact on the Fc binding properties to Fc receptors (e.g., FcγRI, II, III and FcRn) as these receptors engage with the C_{H}2 domains (for the FcγRI, II and III class of receptors) or with the hinge between the C_{H}2 and C_{H}3 domains (e.g. FcRn receptor). Another feature of such antagonists is that both domain antibodies are expected to be spatially close to each other and provided that flexibility is provided by provision of appropriate linkers, these domain antibodies may even form homodimeric species, hence propagating the ‘zipped’ quaternary structure of the Fc portion, which may enhance stability of the antagonist.

Examples of suitable linkers include amino acid sequences which may be from 1 amino acid to 50 amino acids in length, or from 1 amino acid to 40 amino acids, for example, from 1 amino acid to 30 amino acids, or from 1 to 20 amino acids, or from 1 to 10 amino acids, or from 1 to 8 amino acids, or from 1 to 5 amino acids, or from 1 to 3 amino acids, or from 2 to 24 amino acids, or greater than 7 but less than or equal to 10, 15, 20, 25, 30, 35, or 40 amino acids. In one embodiment, the linker is greater than 7 and less than or equal to 50 amino acids in length. In one embodiment, the linker is less than 25 amino acids in length. Such sequences may have their own tertiary structure, for example, a linker of the present invention may comprise a domain antibody. The size of a linker in one embodiment is equivalent to a domain antibody. Suitable linkers may be of a size from 1 to 100 Angstroms, for example may be of a size from 20 to 80 angstroms or for example may be of a size from 20 to 60 angstroms or for example less than 40 angstroms, or less than 20 angstroms, or less than 5 angstroms in length. Where an antagonist comprises two domain antibodies (i.e., a dual dAb), the domains may be attached to the Fc region of an antibody by identical or different linkers.

If a single chain Fc region of an antibody is present in the antagonist, in one embodiment the Fc region is derived from an IgG, such as IgG1, IgG2, IgG3, IgG4, in particular IgG1. In one embodiment, the Fc region is mutated. Such mutations may be in one or more of positions selected from 239, 332 and 330 (IgG1), or the equivalent positions in other IgG isotypes. Examples of suitable mutations are S239D and I332E and A330L (EU index numbering). In an alternative embodiment, the antagonist comprises a heavy chain constant region with an altered glycosylation profile such that the antagonist has enhanced effector function (e.g., enhanced ADCC or enhanced CDC or wherein it has both enhanced ADCC and CDC effector function). Examples of suitable methodologies to produce antigen binding proteins with an altered glycosylation profile are described in WO2003/011878, WO2006/014679 and EP1229125, all of which can be applied to the antagonists of the present invention.

In one embodiment, the TGFβR antagonist comprises a dimer of a dual domain antibody as described herein.

If the antagonists of the present invention comprise additional components to the domain antibody (e.g., a single chain Fc region and/or linker and/or further domain antibody), they may be expressed as a fusion protein or the domain antibody may be expressed separately and connected by another means, such as chemical conjugation using methods well known in the art.

In one embodiment, the TGFβR antagonist binds to TGFβRII with a dissociation constant (Kd) in the range of 10 pM to 50 nM. In a further embodiment, the dissociation constant is in the range of 10 pM to 10 nM, such as 250 pM to 10 nM. In one embodiment, the TGFβR antagonist binds to TGFβRII with high affinity (high potency) and has a dissociation constant of 10 pM to 500 pM. In another embodiment, the TGFβR antagonist binds to TGFβRII with moderate affinity (low potency) and has a dissociation constant of 500 pM to 50 nM, such as 500 pM to 10 nM.

In one embodiment, the TGFβR antagonist also binds to mouse TGFβRII.

In one embodiment, the TGFβR antagonist is an anti-TGFβRII single variable domain described in WO2011/012609 or WO2012/093125, which are herein incorporated by reference.

In one embodiment, the TGFβR antagonist comprises a nucleotide sequence that is at least 70%, such as at least 75%, 80%, 85%, 90%, 95%, 96%, 97%, 98% or 99% identical to at least one nucleotide sequence selected from the group consisting of: SEQ ID NO: 85 to 168, wherein said TGFβR antagonist binds to TGFβRII. In one embodiment, the TGFβR antagonist comprises a nucleotide sequence that is at least 70%, such as at least 75%, 80%, 85%, 90%, 95%, 96%, 97%, 98% or 99% identical to at least one nucleotide sequence selected from the group consisting of: SEQ ID NO: 94 and SEQ ID NO: 156, wherein said TGFβR antagonist binds to TGFβRII.

In one embodiment, the TGFβR antagonist comprises an amino acid sequence that is at least 70%, such as at least 75%, 80%, 85%, 90%, 95%, 96%, 97%, 98% or 99% identical to at least one amino acid sequence selected from the group consisting of: SEQ ID NOs: 1 to 84, wherein said TGFβR antagonist binds to TGFβRII. In one embodiment, the TGFβR antagonist comprises an amino acid sequence that is at least 70%, such as at least 75%, 80%, 85%, 90%, 95%, 96%, 97%, 98% or 99% identical to at least one amino acid sequence selected from the group consisting of: SEQ ID NO: 10 and SEQ ID NO: 72, wherein said TGFβR antagonist binds to TGFβRII.

In a further embodiment, the TGFβR antagonist comprises SEQ ID NO: 10, having up to 5 amino acid substitutions, deletions or additions, in any combination. In an alternative embodiment, the TGFβR antagonist comprises SEQ ID NO: 72, having up to 5 amino acid substitutions, deletions or additions, in any combination.

In one embodiment, said amino acid substitutions, deletions or additions are not within CDR3. In a further embodiment, said amino acid substitutions, deletions or additions are not within any of the CDRs. In one embodiment, said amino acid substitutions are conservative substitutions, for example, substituting one hydrophobic amino acid for an alternative hydrophobic amino acid. For example, leucine may be substituted with valine, or isoleucine

In one embodiment, the TGFβR antagonist comprises SEQ ID NO: 10. In an alternative embodiment, the TGFβR antagonist comprises SEQ ID NO: 72.

### T Cell Receptors and Chimeric Antigen Receptors

In one embodiment, the cell immunotherapy is an immunomodulatory cell expressing a chimeric antigen receptor (CAR) or a modified T cell receptor (TCR) or Natural Killer (NK) cell receptor.

In one embodiment, the cell immunotherapy is a modified TCR. In a further embodiment, the TCR is an αβ heterodimeric TCR or a γδ heterodimeric TCR. In a further embodiment, the TCR is genetically modified, i.e., compared to the natural TCR.

In one embodiment, the cell immunotherapy is a CAR. In a further embodiment, the CAR comprises a target binding domain, a transmembrane domain and an intracellular effector domain. In a yet further embodiment, the CAR additionally comprises a spacer domain between the target binding and transmembrane domains. In a yet further embodiment, the intracellular effector domain additionally comprises a costimulatory domain.

The TCR or target binding domain of the CAR binds to a target, wherein the target is a tumour specific molecule, a pathogen specific molecule (such as a viral molecule), or any other molecule expressed on a target cell population that is suitable to mediate recognition and elimination by a lymphocyte. Therefore, in one embodiment, the CAR or TCR binds to a tumour associated antigen or pathogen antigen. In one embodiment, the target binding domain comprises an antibody, an antigen binding fragment or a ligand. In a further embodiment, the target binding domain is a ligand. In an alternative embodiment, the target binding domain is an antigen binding fragment. In a further embodiment, the antigen binding fragment is a single chain variable fragment (scFv) or a dAb™. In a yet further embodiment, said scFv comprises the light (VL) and the heavy (VH) variable fragment of a target antigen specific monoclonal antibody joined by a flexible linker. In one embodiment, the target binding domain may bind to more than one target, for example two different targets. Such a target binding domain may be derived from a bispecific single chain antibody. For example, Blinatumomab (also known as AMG 103 or MT103) is a recombinant CD19 and CD3 bispecific scFv antibody consisting of four immunoglobulin variable domains assembled into a single polypeptide chain. Two of the variable domains form the binding site for CD19 which is a cell surface antigen expressed on most normal and malignant B cells. The other two variable domains form the binding site for CD3 which is part of the T cell-receptor complex on T cells. These variable domains may be arranged in the CAR in tandem, i.e., two single chain antibody variable fragments (scFv) tethered to a spacer, and transmembrane and signalling domains. The four variable domains can be arranged in any particular order within the CAR molecule (e.g., VL(first target)-VH(first target)-VH(second target)-VL(second target) or VL(second target)-VH(second target)-VH(first target)-VL(first target), etc.).

In one embodiment, the target binding domain and/or spacer domain may comprise a multimerization domain(s), for example as described in WO2015/017214. This enables the signal transduction of the CAR to be controlled through the addition of external agents, such as a chemical drug, which acts a bridging factor between the multimerization domains. Therefore, in one embodiment, the target binding domain and/or spacer domain comprises (a) a first multimerization domain; and (b) a second multimerization domain; wherein a first bridging factor promotes the formation of a polypeptide complex with the bridging factor associated with and disposed between the first and second multimerization domains.

The target binding domain may bind a variety of cell surface antigens, but in one embodiment, the target binding domain binds to a tumour associated antigen. In a further embodiment, the tumour associated antigen is selected from: BCMA, CD19, HER2, prostate stem cell antigen (PSCA), prostate-specific membrane antigen (PSMA), carcinoembryonic antigen (CEA), cancer antigen-125 (CA125), CA19-9, Mucin 1 (MUC-1), tyrosinase, CD34, CD45, CD117, protein melan-A, synaptophysis, CD22, CD27, CD30, CD70, ganglioside G2 (GD2), epidermal growth factor variant III (EGFRvIII), mesothelin, prostatic acid phosphatise (PAP), prostein, Trp-p8, six transmembrane epithelial antigen of the prostate I (STEAP1).

In one embodiment, the target binding domain binds to a pathogen antigen. In a further embodiment, the pathogen antigen is a bacterial antigen, viral antigen, parasitic antigen, protozoan antigen or fungal antigen. In a further embodiment, the pathogen antigen is a bacterial antigen or a viral antigen.

In one embodiment, the transmembrane domain can be derived either from a natural or from a synthetic source. In one embodiment, the transmembrane domain can be derived from any membrane-bound or transmembrane protein. Alternatively the transmembrane domain can be synthetic and can comprise predominantly hydrophobic residues such as leucine and valine.

For example, the transmembrane domain can be the transmembrane domain of CD proteins, such as CD4, CD8, CD3 or CD28, a subunit of the T cell receptor, such as α, β, γ or δ, a subunit of the IL-2 receptor (a chain), a submit of the Low-Affinity Nerve Growth Factor Receptor (LNGFR or p75) (β chain or γ chain), or a subunit chain of Fc receptors. In one embodiment, the transmembrane domain comprises the transmembrane domain of CD4, CD8 or CD28. In a further embodiment, the transmembrane domain comprises the transmembrane domain of CD4 or CD8 (e.g., the CD8 alpha chain, as described in NCBI Reference No.: NP_001139345.1 or a fragment thereof).

In one embodiment, the CAR additionally comprises a spacer domain between the target binding and transmembrane domains. In a further embodiment, the spacer domain is selected from CD8 (e.g., CD8α) or the C_{H}2 and/or C_{H}3 domains of IgG1 or IgG4.

Preferred examples of the effector domain for use in a CAR scaffold can be the cytoplasmic sequences of the natural T cell receptor and co-receptors that act in concert to initiate signal transduction following antigen binding, as well as any derivate or variant of these sequences and any synthetic sequence that has the same functional capability. Effector domains can be separated into two classes: those that initiate antigen-dependent primary activation, and those that act in an antigen-independent manner to provide a secondary or costimulatory signal. Primary activation effector domains can comprise signalling motifs which are known as immunoreceptor tyrosine-based activation motifs (ITAMs). ITAMs are well defined signalling motifs, commonly found in the intracytoplasmic tail of a variety of receptors, and serve as binding sites for syk/zap70 class tyrosine kinases. Examples of ITAMs used in the invention can include, as non-limiting examples, those derived from CD3ζ, FcRγ, FcRβ, FORε, CD3γ, CD3δ, CD3ε, CD5, CD22, CD79a, CD79b and CD66d. In one embodiment, the intracellular effector domain comprises a CD3ζ signalling domain (also known as CD247). Natural TCRs contain a CD3ζ signalling molecule, therefore the use of this effector domain is closest to the TCR construct which occurs in nature. In a further embodiment, the CD3ζ signalling domain comprises the sequence as described in NCBI Reference No.: NP_932170, or a fragment thereof that has activating or stimulatory activity.

Activating NK cell receptors specific for classic MHC class I molecules, non-classic MHC class I molecules or MHC class I-related molecules have been described (Bakker, et al., (2000) Hum. Immunol. 61:18-27). One such receptor is NKG2D (natural killer cell group 2D) which is a C-type lectin-like receptor expressed on NK cells, γδTcR+ T cells, and CD8+αβ−TcR+ T cells (Bauer, et al., (1999) Science 285:727-730). NKG2D is associated with the transmembrane adapter protein DAP10 (Wu, et al., (1999) Science 285:730-732), whose cytoplasmic domain binds to the p85 subunit of the PI-3 kinase.

The capacity of NK cells to kill tumour cells depends on the combined effect of inhibitory and stimulatory signals delivered through surface receptors. The interaction between some members of the killer immunoglobulin-like receptor (KIR) family on NK cells and cognate HLA Class I molecules on potential target cells produces inhibitory signals, a mechanism that prevents the killing of autologous cells. Signals from activating receptors are triggered by ligands expressed predominantly by virally-infected and tumour cells; hence, these receptors are central to the capacity of NK cells to recognize and lyse unhealthy cells.

Therefore, other examples of the effector domain for use in a CAR scaffold can be based on NK cell activating receptors, as well as any derivate or variant of these sequences and any synthetic sequence that has the same functional capability. Many NK cell activating receptors belong to the Ig superfamily (IgSF) (such receptors also may be referred to as Ig-like receptors or “ILRs” herein). Activating ILR NK receptors (AILRs) include, e.g., CD2, CD16, CD69, DNAX accessory molecule-1 (DNAM-1), 2B4, NK1.1; killer immunoglobulin (Ig)-like activating receptors (KARs); ILTs/LIRs; and natural cytotoxicity receptors (NCRs), such as NKp44, NKp46, and NKp30. Several other activating receptors belong to the CLTR superfamily (e.g., NKRP-1, CD69; CD94/NKG2C and CD94/NKG2E heterodimers, NKG2D homodimer, and in mice, activating isoforms of Ly49, such as Ly49A-D). Still other activating receptors (e.g., LFA-1 and VLA-4) belong to the integrin protein superfamily and other activating receptors may have even other distinguishable structures. Many activating receptors possess extracellular domains that bind to MHC-I molecules, and cytoplasmic domains that are relatively short and lack the immunoreceptor tyrosine-based inhibition motif (ITIM) signalling motifs characteristic of inhibitory NK receptors. The transmembrane domains of these receptors typically include a charged amino acid residue that facilitates their association with signal transduction-associated molecules, e.g., CD3ζ, FcεRIγ, DAP12, and DAP10 (2B4, however, appears to be an exception to this general rule), which contain short amino acid sequences termed an “immunoreceptor tyrosine-based activating motif” (ITAMs) that propagate NK cell-activating signals. Receptor 2B4 contains 4 Immunoreceptor Tyrosine-based Switch Motifs (ITSMs) in its cytoplasmic tail. ITSM motifs can also be found in NKCARs CS1/CRACC and NTB-A. The cytoplasmic domains of 2B4 and SLAM contain two or more unique tyrosine-based motifs that resemble motifs presents in activating and inhibitory receptors and can recruit the SH2-domain containing proteins SHP-2 and SLAM-associated protein (SAP).

As described herein, effector domains may also provide a secondary or costimulatory signal. T cells additionally comprise costimulatory molecules which bind to cognate costimulatory ligands on antigen presenting cells in order to enhance the T cell response, for example by increasing proliferation activation, differentiation and the like. Therefore, in one embodiment, the intracellular effector domain additionally comprises a costimulatory domain. In a further embodiment, the costimulatory domain comprises the intracellular domain of a costimulatory molecule, selected from CD28, CD27, 4-1BB (CD137), OX40 (CD134), ICOS (CD278), CD30, CD40, PD-1 (CD279), CD2, CD7, NKG2C (CD94), B7-H3 (CD276) or any combination thereof. In a yet further embodiment, the costimulatory domain comprises the intracellular domain of a costimulatory molecule, selected from CD28, CD27, 4-1BB, OX40, ICOS or any combination thereof. In a yet further embodiment, the costimulatory domain comprises CD28, e.g. as described in NCBI Reference No.: NP_006130, or a fragment thereof that has activating or stimulatory activity.

### Polynucleotides and Expression Vectors

According to a further aspect of the invention, there is provided a polynucleotide comprising a sequence that encodes a TGFβ Receptor (TGFβR) antagonist which comprises a domain antibody, and a sequence that encodes a chimeric antigen receptor (CAR) or a T cell receptor (TCR).

The polynucleotides of the present invention may be combined with other DNA sequences, such as promoters and/or enhancers, untranslated regions (UTRs), signal sequences, Kozak sequences, polyadenylation sequences, restriction enzyme sites, multiple cloning sites, internal ribosomal entry sites (IRES), recombinase recognition sites (e.g., LoxP, FRTand Attsites), termination codons, transcriptional termination signals and polynucleotides encoding self-cleaving polypeptides.

The sequences may be part of the same, or different expression cassettes. For example, in one embodiment, the sequence that encodes a TGFβR antagonist and sequence that encodes a CAR or a TCR are each operably linked to a promoter element (i.e., the TGFβR antagonist and CAR/TCR are encoded by separate expression cassettes). In an alternative embodiment, the sequence that encodes a TGFβR antagonist and sequence that encodes a CAR or a TCR are controlled by a single promoter (i.e., they are part of the same expression cassette). In this embodiment, it will be understood that the TGFβR antagonist and CAR/TCR are expressed as one long sequence, therefore it would be suitable to include an internal ribosome entry site (IRES) or a polynucleotide encoding a self-cleaving peptide, e.g., a P2A peptide, to ensure that each sequence is translated separately.

In one embodiment, the promoter element is an inducible or constitutive promoter. If the sequences are present in separate expression cassettes, it will be understood that the promoter element used for each expression cassette may be different.

The polynucleotide may be present in an expression cassette or expression vector (e.g., a plasmid for introduction into a bacterial host cell, or a viral vector such as a lentivirus for transfection of a mammalian host cell). Therefore, according to a further aspect of the invention, there is provided an expression vector comprising the polynucleotide described herein.

In one embodiment, the expression vector is a viral vector. In a further embodiment, the viral vector is derived from, or selected from, a retroviral vector or an adeno-associated viral (AAV) vector.

In one embodiment, the retroviral vector is derived from, or selected from, a lentivirus, alpha-retrovirus, gamma-retrovirus or foamy-retrovirus, such as a lentivirus or gamma-retrovirus, in particular a lentivirus. In a further embodiment, the retroviral vector particle is a lentivirus selected from the group consisting of HIV-1, HIV-2, SIV, FIV, EIAV and Visna. Lentiviruses are able to infect non-dividing (i.e., quiescent) cells which makes them attractive vectors for gene therapy. In a yet further embodiment, the retroviral vector particle is HIV-1 or is derived from HIV-1. The genomic structure of some retroviruses may be found in the art. For example, details on HIV-1 may be found from the NCBI Genbank (Genome Accession No. AF033819). HIV-1 is one of the best understood retroviruses and is therefore often used as a viral vector.

As a non-limiting example, the TGFβR antagonist and CAR/TCR can be introduced as transgenes encoded by an expression vector as described herein. The expression vector can also contain a selection marker which provides for identification and/or selection of cells which received said vector.

### Immunomodulatory Cells

According to a further aspect of the invention, there is provided an immunomodulatory cell comprising the polynucleotide or expression vector as described herein. In one embodiment, the immunomodulatory cell may be a human immunomodulatory cell.

The term “immunomodulatory cell” refers to a cell of hematopoietic origin functionally involved in the modulation (e.g., the initiation and/or execution) of the innate and/or adaptive immune response. Said immunomodulatory cell according to the present invention can be derived from a stem cell. The stem cells can be adult stem cells, non-human embryonic stem cells, more particularly non-human stem cells, cord blood stem cells, progenitor cells, bone marrow stem cells, induced pluripotent stem cells, totipotent stem cells or hematopoietic stem cells. Said immunomodulatory cell can also be a dendritic cell, a killer dendritic cell, a mast cell, a natural killer (NK) cell, a B cell or a T cell. The T cell may be selected from the group consisting of inflammatory T-lymphocytes, cytotoxic T-lymphocytes, regulatory T-lymphocytes or helper T-lymphocytes, or a combination thereof. Therefore, in one embodiment, the immunomodulatory cell is derived from an inflammatory T-lymphocyte, cytotoxic T-lymphocyte, regulatory T-lymphocyte or helper T-lymphocyte. In another embodiment, said cell can be derived from the group consisting of CD4⁺ T-lymphocytes and CD8⁺ T-lymphocytes. In one embodiment, the immunomodulatory cell is derived from a T cell (such as an inflammatory T-lymphocyte, cytotoxic T-lymphocyte, regulatory T-lymphocyte or helper T-lymphocyte), natural killer cell, or a pluripotent stem cell from which lymphoid cells may be differentiated. In a further embodiment, the immunomodulatory cell is derived from a T cell or a natural killer cell.

In one embodiment, the immunomodulatory cell is derived from a natural killer cell. Natural killer (NK) cells can recognize tumour cells as targets and as such may be useful for immunotherapy of cancer (Vivier et al., 2011, Science 331:44-49; Ruggeri et al., 2002, Science 295:2097-2100; Cooley et al., 2010, Blood 116:2411-2419; Miller et al., 2005, Blood 105:3051-3057; Rubnitz et al., 2010, J Clin Oncol. 28:955-959). Infusions of NK cells have been used to treat patients with various forms of cancer (Caligiuri, 2008, Blood 112(3):461-469). Methods are available that make it possible to obtain a large number of human NK cells that demonstrate a higher anti-tumour capacity than that of non-expanded NK cells (see U.S. Pat. No. 7,435,596; Imai et al., 2005, Blood 106:376-83; Fujisaki et al., 2009, Cancer Res. 69: 4010-4017; Cho et al., 2010, Clin Cancer Res. 16:3901-3909).

Prior to expansion and genetic modification of the cells of the invention, a source of cells can be obtained from a subject through a variety of non-limiting methods. Cells can be obtained from a number of non-limiting sources, including peripheral blood mononuclear cells (PBMCs), bone marrow, lymph node tissue, cord blood, thymus tissue, tissue from a site of infection, ascites, pleural effusion, spleen tissue, and tumours. In certain embodiments of the present invention, any number of T cell lines available and known to those skilled in the art, may be used. In another embodiment, said immunomodulatory cell can be derived from a healthy donor or a diseased donor, such as a patient diagnosed with cancer or an infection. In another embodiment, said immunomodulatory cell is part of a mixed population of cells which present different phenotypic characteristics.

In one embodiment, immunomodulatory cells can be obtained from a unit of blood collected from a subject using any number of techniques known to a person skilled in the art, such as apheresis, centrifugation and/or sedimentation. Collected cells can then be activated and/or expanded before or after genetic modification (i.e., to express the CAR/TCR) using methods known in the art, e.g., contact with an anti-CD3 antibody, an anti-CD2 antibody and/or a protein kinase C activator.

In one embodiment, the immunomodulatory cell is autologous (i.e., derived from the patient's own immune cells). In an alternative embodiment, the immunomodulatory cell is allogeneic (i.e., derived from another individual). It will be understood that in order to prevent the allogeneic cells from being rejected by the patient, they would either need to be derived from a compatible donor or modified to ensure no antigens are present on the cell surface which would initiate an unwanted immune response.

It will be understood that the immunomodulatory cells may express the TGFβR antagonist and chimeric antigen receptor or T cell receptor transiently or stably/permanently (depending on the transfection method used and whether the polynucleotide has integrated into the immunomodulatory cell genome or not). Once the CAR/TCR has been introduced into the immunomodulatory cell, said cell may be referred to as a “transformed immunomodulatory cell”.

### Uses

According to a further aspect of the invention, there is provided the pharmaceutical composition described herein for use in therapy. In one embodiment, the pharmaceutical composition is for use in the treatment of a disease selected from: cancer, an autoimmune disease, or an infection. In a further embodiment, the pharmaceutical composition is for use in the treatment of cancer. Compositions of the present invention are particularly well suited for the treatment of cancer because the TGFβR antagonist prevents TGFβR signalling and therefore overcomes the immunosuppressive effects of the tumour microenvironment to prolong the effect of the cell immunotherapy.

According to a further aspect of the invention, there is provided the immunomodulatory cell described herein for use in therapy. In one embodiment, therapy comprises administration of the immunomodulatory cell to a human subject in need of such therapy.

In one embodiment, the immunomodulatory cell is for use in the treatment of a disease selected from: cancer, an autoimmune disease or an infection. In a further embodiment, the immunomodulatory cell is for use in the treatment of cancer.

According to a further aspect of the invention, there is provided use of the pharmaceutical composition described herein, or the immunomodulatory cell described herein, in the manufacture of a medicament for use in therapy. In one embodiment, the manufacture of the medicament is for use in the treatment of a disease selected from: cancer, an autoimmune disease, or an infection.

### Methods

According to a further aspect of the invention, there is provided a method for producing an antigen-specific immunomodulatory cell, the method comprising introducing into an immunomodulatory cell the polynucleotide or expression vector as defined herein. It will be understood by a person skilled in the art that the polynucleotides and expression vectors of the present invention comprise CAR/TCRs which are antigen specific and therefore expression of these molecules on the surface of an immunomodulatory cell confers that antigen-specificity to the cell.

Polypeptides may be synthesized in situ in the cell as a result of the introduction of polynucleotides encoding said CAR/TCR and TGFβR antagonist into the cell. Alternatively, said polypeptides could be produced outside the cell and then introduced thereto. Methods for introducing a polynucleotide construct into cells are known in the art and including, as non-limiting examples, stable transformation methods wherein the polynucleotide construct is integrated into the genome of the cell or transient transformation methods wherein the polynucleotide construct is not integrated into the genome of the cell. Said polynucleotides may be introduced into a cell by, for example, recombinant viral vectors (e.g., retroviruses, adenoviruses), liposomes and the like. For example, transient transformation methods include for example microinjection, electroporation or particle bombardment. The polynucleotides may be included in vectors, more particularly plasmids or viruses, in view of being expressed in cells.

It will be understood that methods of the invention may be performed in vitro, ex vivo or in vivo.

According to a further aspect of the invention, there is provided a method for producing an antigen-specific immunomodulatory cell, the method comprising introducing into an immunomodulatory cell the expression vector as defined herein.

In one embodiment, the method comprises transfecting the expression vector as described herein into the immunomodulatory cell. The terms “transfection”, “transformation” and “transduction” as used herein, may be used to describe the insertion of the expression vector into the target cell. Insertion of a vector is usually called transformation for bacterial cells and transfection for eukaryotic cells, although insertion of a viral vector may also be called transduction. The skilled person will also be aware of the different non-viral transfection methods commonly used, which include, but are not limited to, the use of physical methods (e.g., electroporation, cell squeezing, sonoporation, optical transfection, protoplast fusion, impalefection, magnetofection, gene gun or particle bombardment), chemical reagents (e.g., calcium phosphate, highly branched organic compounds or cationic polymers) or cationic lipids (e.g., lipofection). Many transfection methods require the contact of solutions of plasmid DNA to the cells, which are then grown and selected for a marker gene expression.

### Methods of Treatment

According to a further aspect of the invention, there is provided a method of treatment comprising administering the expression vector as defined herein, to a subject.

According to a further aspect of the invention, there is provided a method of treatment comprising administering the immunomodulatory cell as defined herein, to a subject.

According to a further aspect of the invention, there is provided a method of treatment comprising administering to a subject:

(a) a cell immunotherapy, and

(b) a TGFβ receptor (TGFβR) antagonist which comprises a domain antibody.

In one embodiment, the cell immunotherapy is an immunomodulatory cell expressing a chimeric antigen receptor (CAR) or a modified T cell receptor (TCR).

In one embodiment, the TGFβR antagonist is secreted from the immunomodulatory cell. In this embodiment, the sequence encoding the TGFβR antagonist could be attached to a secretion leader sequence so that it will be secreted from the immunomodulatory cell via the secretory pathway (i.e., through the endoplasmic reticulum, the Golgi apparatus, and as a vesicle which fuses to the cell plasma membrane, thus releasing the antagonist outside the cell). Furthermore, in this embodiment, the sequence encoding the cell immunotherapy (e.g., CAR or TCR) and the TGFβR antagonist are both introduced into the immunomodulatory cell, i.e., they are co-expressed by the immunomodulatory cell. The immunomodulatory cell is targeted to the site of disease due to the antigen specificity of the CAR/TCR, therefore the advantage of this embodiment is that it ensures the TGFβR antagonist is expressed only at the site of disease and therefore minimises any adverse systemic effects.

In an alternative embodiment, the TGFβR antagonist and cell immunotherapy are administered separately. In this embodiment, only the sequence encoding the CAR/TCR is introduced/transfected into the immunomodulatory cell and the TGFβR antagonist is administered as part of a separate composition. In this embodiment, the TGFβR antagonist and cell immunotherapy may be administered either sequentially or simultaneously in separate or combined pharmaceutical formulations by any convenient route.

When administration is sequential, either the TGFβR antagonist or the cell immunotherapy may be administered first. In one embodiment, the cell immunotherapy is administered first. In an alternative embodiment, the TGFβR antagonist is administered first.

In one embodiment, when the TGFβR antagonist and cell immunotherapy are administered separately, the TGFβR antagonist is administered directly to the site of disease, i.e., administered locally. For example, if the disease to be treated is cancer, the TGFβR antagonist is administered directly to the tumour. In an alternative example, if the disease to be treated is an infection, then the TGFβR antagonist is administered directly to the site of infection. Administering the TGFβR antagonist directly to the site of disease has the advantage of minimising the systemic effects of the antagonist.

In one embodiment, the method is used to treat cancer. In a further embodiment, the cancer is selected from: blood, bone marrow, lymph, lymphatic system, bladder, breast, colon, cervix, esophagus, kidney, large intestine, lung, oral cavity, ovary, pancreas, prostate, rectum, skin or stomach. In a yet further embodiment, the cancer is a blood cancer, for example selected from the group consisting of: B cell leukaemia, multiple myeloma (MM), acute lymphoblastic leukaemia (ALL), chronic lymphocytic leukaemia (CLL) and non-Hodgkin's lymphoma.

When the method described herein is used to treat cancer, in one embodiment, the method reduces the number of tumour cells, reduces the tumour size and/or eradicates the tumour in the subject.

In one embodiment, the method is used to treat an autoimmune disease. Autoimmune diseases arise from an abnormal immune response of the body against substances and tissues normally present in the body. This can result in the damage or destruction of tissues, or altered organ growth or function. Examples of autoimmune diseases include, but are not limited to: diabetes mellitus Type 1, arthritis (including juvenile, psoriatic, reactive, and rheumatoid arthritis), psoriasis, multiple sclerosis, vasculitis, alopecia areata, pernicious anaemia, glomerulonephritis, autoimmune hepatitis, autoimmune pancreatitis, ulcerative colitis, systemic lupus erythematosus, Graves' disease, Guillain-Barré syndrome, Sjogren's syndrome, Celiac disease, Crohn's disease and Wegener's syndrome.

In one embodiment, the method is used to treat an infection. An infection can be caused by a pathogen, such as a bacteria, virus, parasite, protozoa or fungi. In a further embodiment, the infection is a viral or bacterial infection.

In one embodiment, the subject is a mammal. In a further embodiment, the mammal is selected from the group consisting of: a human, a mouse, a primate, a cow, a pig, a horse, a sheep, a cat, and a dog. In a yet further embodiment, the subject is a human.

According to a further aspect of the invention, there is provided a method of increasing (or lengthening) survival of a subject having cancer, comprising administering to a subject:

(a) a cell immunotherapy, and

(b) a TGFβ receptor (TGFβR) antagonist which comprises a domain antibody.

According to a further aspect of the invention, there is provided a method of reducing tumour burden in a subject, comprising administering to a subject:

(a) a cell immunotherapy, and

(b) a TGFβ receptor (TGFβR) antagonist which comprises a domain antibody.

It will be understood by a person skilled in the art that the compositions and methods described herein can be used in combination with any further treatments or therapies in order to alleviate a disease.

### Kits

According to a further aspect of the invention, there is provided a kit for the treatment of a disease selected from: cancer, an autoimmune disease or infection, comprising the pharmaceutical composition as defined herein.

According to a further aspect of the invention, there is provided a kit for the treatment of a disease selected from: cancer, an autoimmune disease or infection, comprising the polynucleotide as defined herein.

According to a further aspect of the invention, there is provided a kit for the treatment of a disease selected from: cancer, an autoimmune disease or infection, comprising the expression vector as defined herein.

According to a further aspect of the invention, there is provided a kit for the treatment of a disease selected from: cancer, an autoimmune disease or infection, comprising the immunomodulatory cell as defined herein.

In one embodiment, the kits described herein additionally comprise written instructions for using said composition, polynucleotide, expression vector or immunomodulatory cell for the treatment of a subject having cancer, an autoimmune disease or infection.

It will be understood that the embodiments described herein may be applied to all aspects of the invention. Furthermore, all publications, including but not limited to patents and patent applications, cited in this specification are herein incorporated by reference as though fully set forth.

### EXAMPLES

The disclosure is further described in detail by reference to the following experimental (Example 2) and prophetic examples (Examples 1, 3, 4 and 5). These examples are provided for the purposes of illustration only, and are not to be interpreted as limiting the scope of the invention. The invention should in no way be construed as being limited to the following examples, but rather, should be construed to encompass any and all variations which will become evident as a result of the teaching provided herein. Without further description, it is believed that one of ordinary skill in the art can, using the preceding description and the following examples, make and utilize the compounds of the present invention and practice the claimed methods.

### Example 1: Choice of TGFβR Antagonist

Use of an anti-TGFβR domain antibody (single variable domain or dAb™) offers advantages for enhancing the efficacy of immune-receptor transgenic (or naturally arising) adoptive cell therapies for cancer such as those based on T cells or NK cells. These could include Chimeric Antigen Receptor (CAR) or T cell Receptor (TCR) transgenic T or NK cells.

WO2011/012609 and WO2012/093125 describe anti-TGFβRII domain antibodies developed to treat Keloid scarring. The domain antibody comprising the amino acid sequence of SEQ ID NO: 72 has been demonstrated to be a potent inhibitor of TGFβ signalling in human and macaque cells, and has passed GLP pre-clinical safety assessments when delivered as a therapeutic protein. This single variable domain has also been shown to have a sub nanomolar (nM) range IC₅₀ against TGFβ signalling in a reporter gene assay and a nM range IC₅₀ against proximal signalling event (SMAD phosphorylation) in human PBMCs.

A further advantage for using this single variable domain is that it has a short in vivo half-life due to glomerular filtration, which is beneficial for the present invention since many of the side effects of checkpoint antibodies stem from their body wide activity and long serum half-lives following IV dosing.

The use of domain antibodies is also advantageous because they have a higher volume of distribution than monoclonal antibodies which is thought to help facilitate penetration of solid tumour masses, especially when delivered by a T cell homing into the tumour site.

In order to confirm antigen binding of the antagonist, any suitable assay known in the art may be used, for example the assays described in WO2011/012609 and WO2012/093125.

### Example 2: Efficacy of TGFβR Antagonist in Preventing TGFβ Signalling

Experiments were conducted to determine the efficacy of TGFβR antagonist in preventing the effect of TGFβ on cytokine production and surface marker expression in human CD4⁺ T-lymphocytes and CD8⁺ T-lymphocytes activated with anti-CD3 and anti-CD28 antibodies.

### Plate Coating

Anti-CD3 (OKI clone) and anti-CD28 (CD28.2 clone) antibodies were diluted to a final concentration of 1 pg/mL and 3 pg/mL, respectively, in phosphate-buffered saline (PBS). Flat bottom 96-well plates (Falcon #BD351172) were co-coated with 200 μL of each antibody per well and incubated overnight at 4° C. The plates were washed 3x with PBS prior to plating with the activated T-lymphocytes.

CD4⁺ and CD8⁺ T-lymphocyte Isolation

CD4⁺ T-lymphocytes and CD8⁺ were separately purified from whole blood from healthy individuals via immunomagnetic negative selection (StemCell Technologies #19662 and #19663). Cell pellets were resuspended in serum free media (AIM V®, Gibco), and cells were subsequently counted and checked for viability. Cells were plated (2×10⁵ cells/well/200 μL serum free media) onto the pre-coated 96-well plates described previously.

### T-Cell Stimulation

Cells were incubated with TGFβR antagonist dAb comprising the amino acid sequence of SEQ ID NO: 72 (5 nM or 50 nM) for 30 minutes at 37° C. and 5% CO₂. Human TGFβ (Peprotech #100-21C) (1, 10, 50, or 100 ng/mL) was added and the plates were incubated for 24, 48, and 72 hours at 37° C. and 5% CO₂. The media was aspirated for supernatant analysis by MSD using human cytokine (IL-2, IL-6, IL-10, and IL17) and human interferon gamma (IFN-γ) assay kits (Meso Scale Diagnostics (MSD)) for each time point. Triplicate cultures for each time point were combined into one well for flow analysis and then split for required panels. Flow staining and MSD analyses were conducted.

### Flow Cytometry for Baseline Characterization and T-Cell Staining

Approximately 1×10⁵ CD4⁺ or CD8⁺ cells were added per well to 96-well 2 mL deep well plates (Axygen). Plates were incubated with 10 μL/well of human Fc blocking solution (Miltenyi Biotec) for 10-15 minutes in the dark at 4° C. Surface expression was evaluated for CD103 (PE-Cy7 labelled Ber-ACT8), CXCR4 (PE labelled 12G5), OX40 (BV421 labelled Ber-ACT35), and PD1 (BV510 labelled EH12.2H7), by flow cytometry.

### MSD Analysis

Standard protocols for the MSD Human Cytokine Kit and human interferon gamma assay kits were followed.

### Results

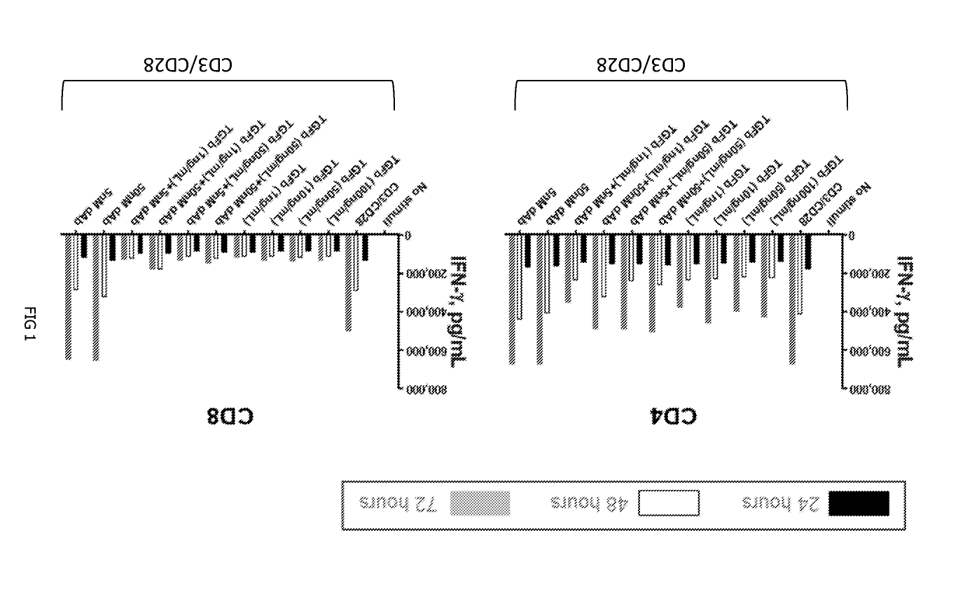

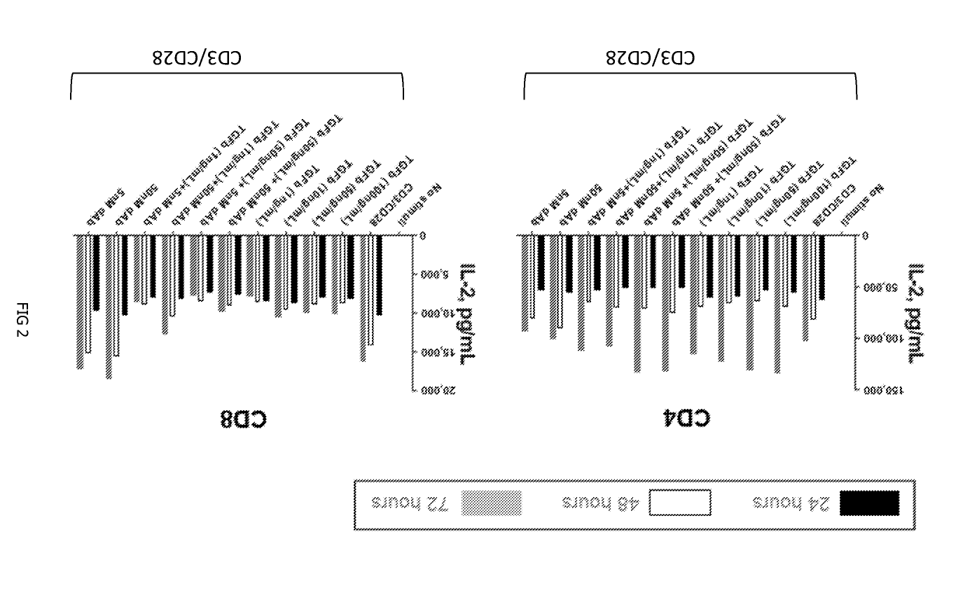

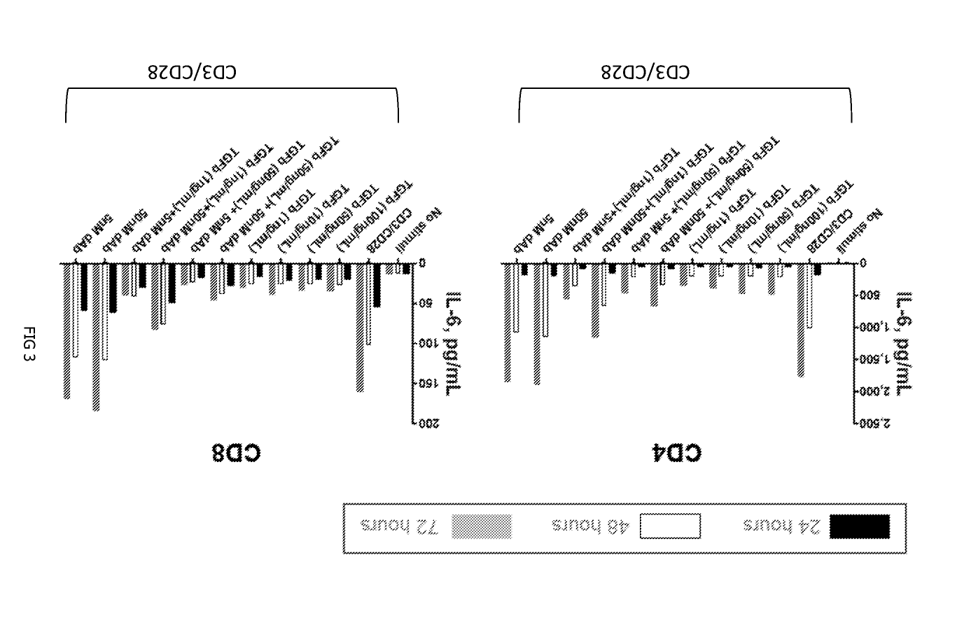

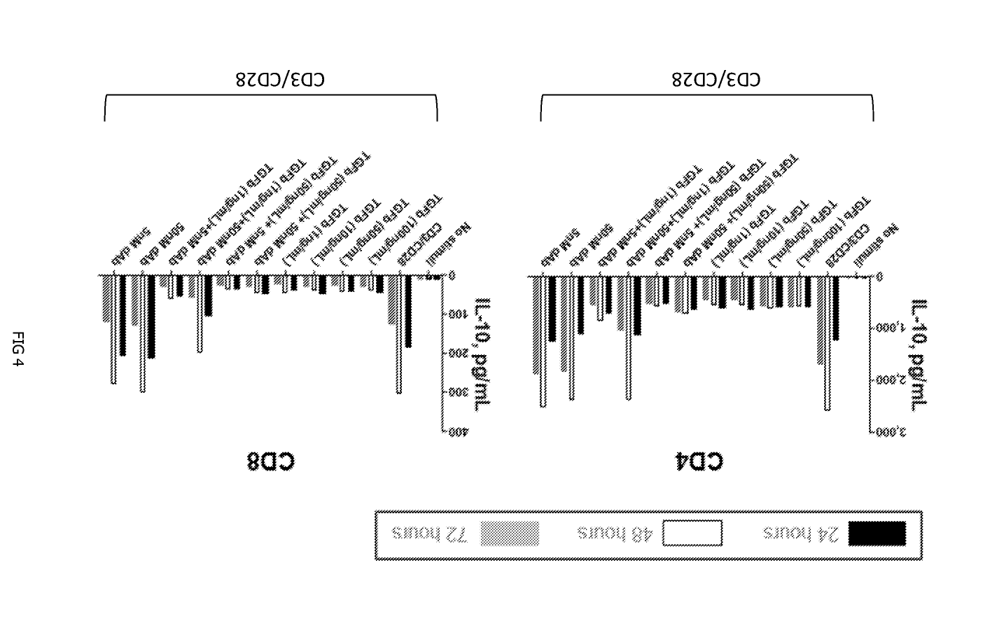

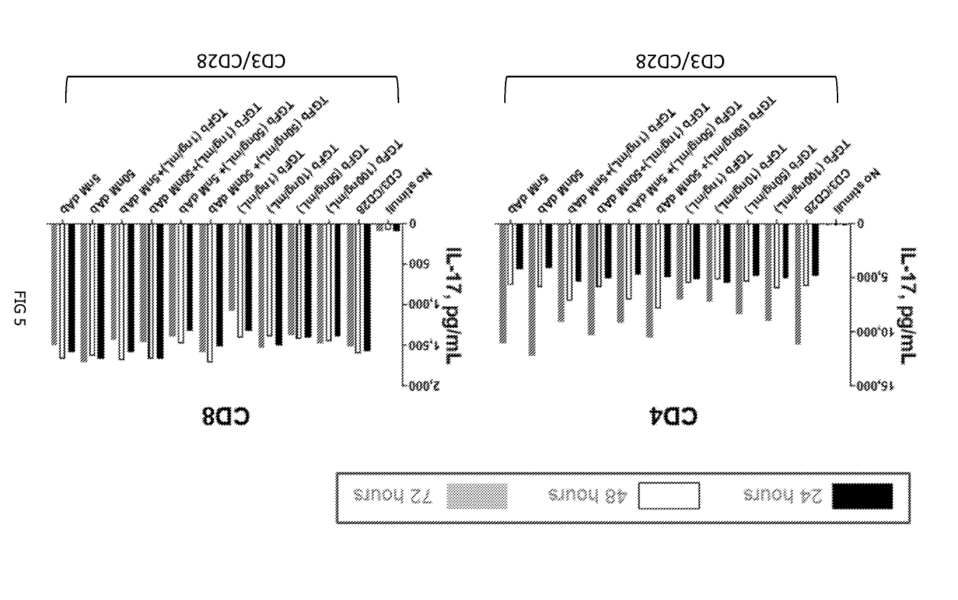

IFN-γ, IL-2, IL-6, IL-10, and IL-17 production by the CD4⁺ and CD8⁺ T-lymphocytes was induced by CD3/CD28 over isotype control in a time-dependent manner. Addition of TGFβ (1-100 ng/mL concentration range) reduced production of these cytokines over CD3/CD28, with concentrations of TGFβ at 1 ng/mL appearing to be saturating. Addition of TGFβR antagonist dAb at 50 nM had the following effects: 1. Enhanced IFN-γ production in the presence of 1 ng/mL TGFβ with comparable efficacy at 48 and 72 hour time points for both CD4⁺ and CD8⁺ T-lymphocytes; 2. Enhanced IL-2 production in the presence of 1 ng/mL TGFβ with stronger efficacy at 72 hours in CD8⁺ lymphocytes, but not in CD4⁺ T-lymphocytes; 3. Enhanced IL-6 production in the presence of 1 ng/mL TGFβ with comparable efficacy at all three points for both CD4⁺ and CD8⁺ T-lymphocytes; 4. Enhanced IL-10 production in the presence of 1 ng/mL TGFβ with stronger efficacy at 48 hours for both CD4⁺ and CD8⁺ T-lymphocytes; and 5. Enhanced IL-17 production in the presence of 1 ng/mL TGFβ with stronger efficacy at 72 hours for both CD4⁺ and CD8⁺ T-lymphocytes. These results are illustrated in FIGS. 1-5.

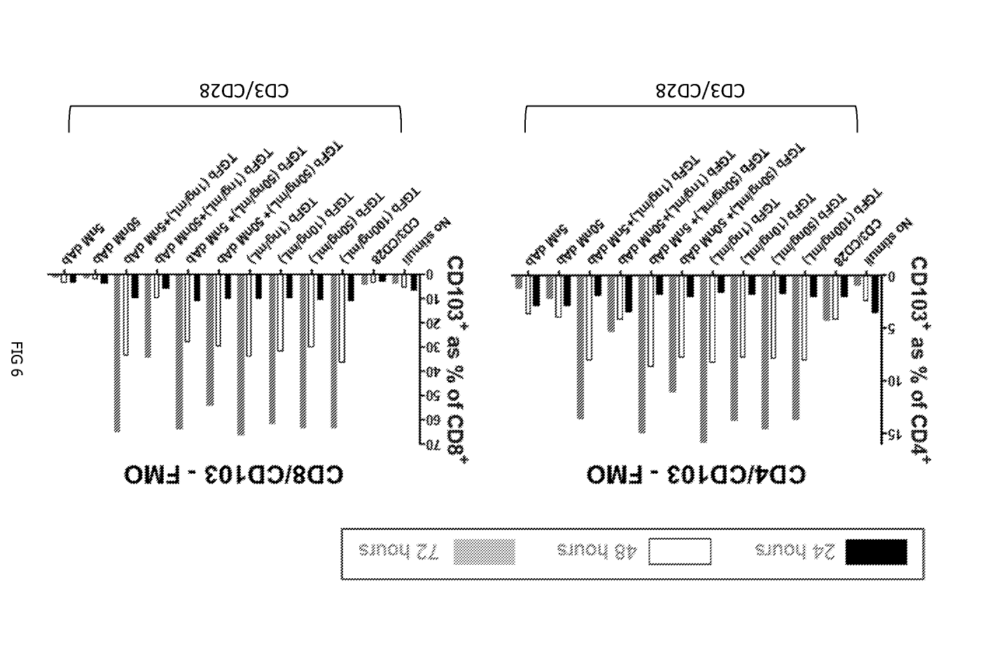

Cell surface expression of CD103 was induced specifically by TGFβ in time-dependent manner starting at 48 hours. Addition of TGFβR antagonist dAb at 50 nM decreased CD103 expression in the presence of 1 ng/mL TGFβ with strong efficacy at 72 hours in CD4⁺ and 48 hours in CD8⁺ T-lymphocytes (FIG. 6).

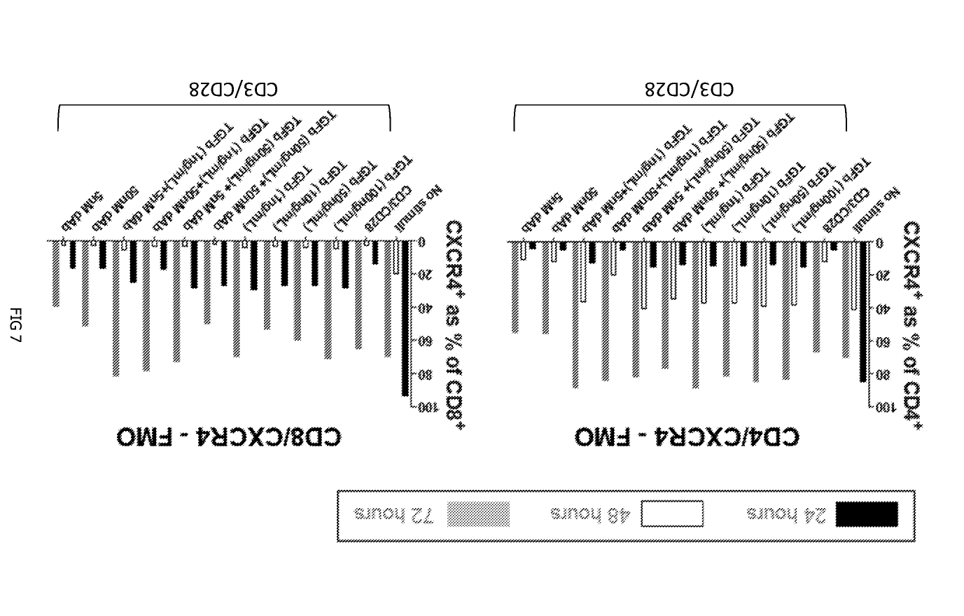

Cell surface expression of CXCR4 was reduced in the presence of CD3/CD28 stimuli at 24 and 48 hour time points. Addition of TGFβ (1-100 ng/mL concentration range) enhanced CXCR4 expression over CD3/CD28 at 24h and 48h time points. Addition of TGFβR antagonist dAb at 50 nM decreased CXCR4 expression in the presence of 1 ng/mL TGFβ with strongest efficacy at 24 hours in both CD4⁺ and CD8⁺ T-lymphocytes (FIG. 7).

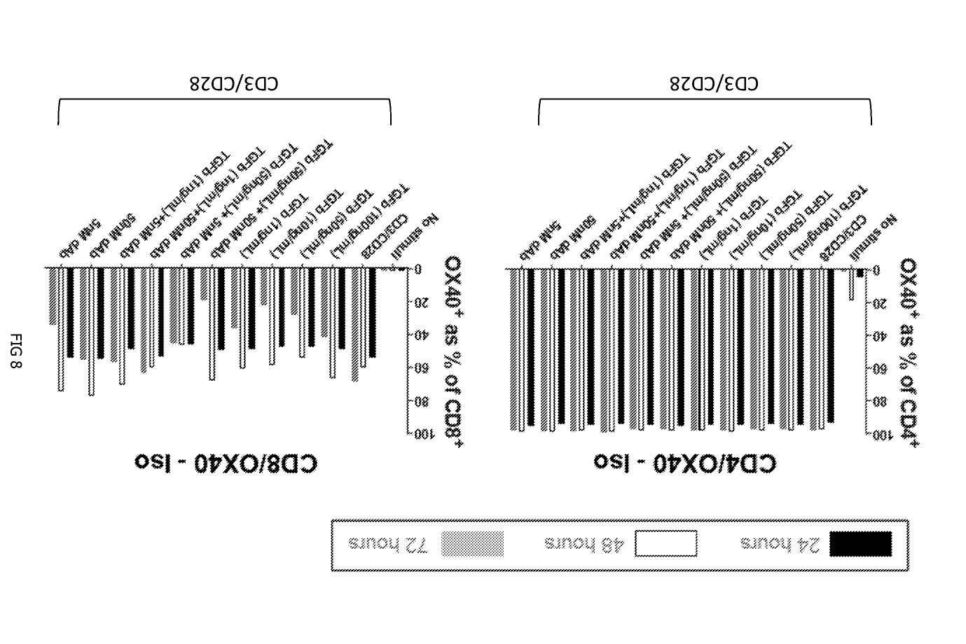

Cell surface expression of OX40 was dramatically induced in the presence of CD3/CD28 stimuli at all three time points. Addition of TGFβ (1-100 ng/mL concentration range) in the presence of CD3/CD28 reduced OX40 expression over CD3/CD28 alone at 72 hours in CD8⁺ T-lymphocytes only. Addition of TGFβR antagonist dAb at 50 nM had a slight recovery effect (increase) on OX40 expression in the presence of 1 ng/mL TGFβ at 72 hours in CD8⁺ T-lymphocytes (FIG. 8).

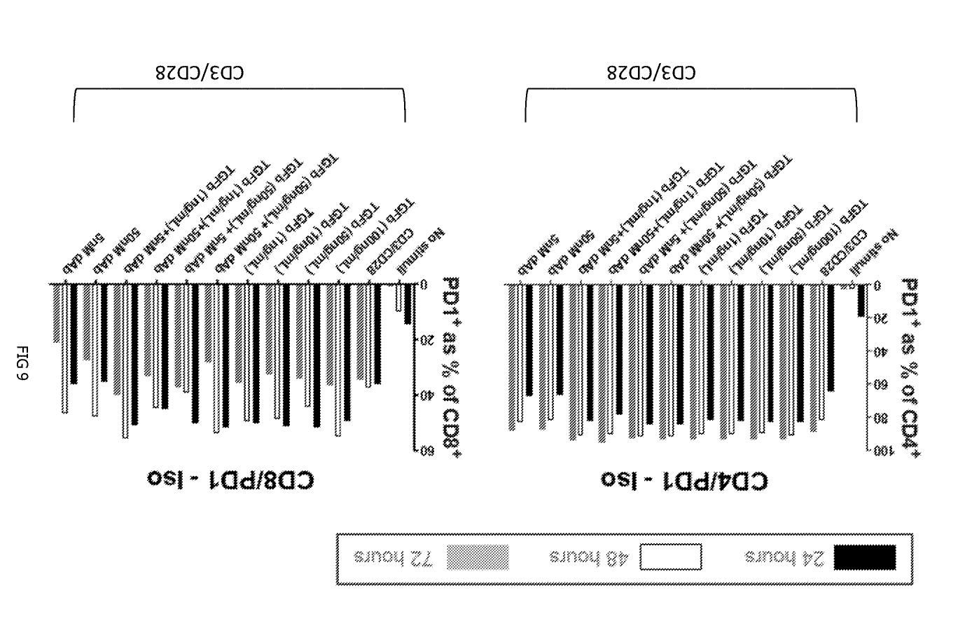

Cell surface expression of PD1 was induced in the presence of CD3/CD28 stimuli at all three time points. Addition of TGFβ (1-100 ng/mL concentration range) enhanced in the presence of CD3/CD28 slightly enhanced PD1 over CD3/CD28 alone at 24 and 48 hour time points in CD8⁺ T-lymphocytes only. Addition of TGFβR antagonist dAb at 50 nM had minimal effect on PD1 expression in the presence of 1 ng/mL TGFβ at 24 and 48 hours in CD8⁺ T-lymphocytes (FIG. 9).

### Example 3: Construction of the CAR

If the TGFβR antagonist is used in combination with a CAR, then a generic CAR architecture may be used, for example comprising a target-specific scFv, CD8 transmembrane domain, CD28 intracellular domain and CD3zeta (CD3ζ) signalling domain. The entire construct may be constructed by synthesising the individual DNA fragments of each component of the CAR molecule and incorporating appropriate restriction sites in the DNA sequences. Standard molecular biology protocols are followed to PCR amplify, restriction enzyme digest, purify and ligate DNA fragments into expression vectors.

In order to confirm antigen binding of the scFv used in the CAR molecule, soluble scFv fragments produced and purified from mammalian expression systems may be subjected to in vitro affinity determination to their antigen. A dilution series of scFv protein in HBS-EP buffer is injected over a BIAcore T200 chip surface previously coated with antigen at an appropriate ‘Response Unit Density’ and the sensogram recorded. Analysis of the binding kinetics can be assisted by the proprietary software using an appropriate fitting model (mostly 1:1 binding). This affinity data can be used to confirm suitability of scFv fragments to be used in the CAR construct.

### Example 4: Expression of CAR/TCR and TGFβR Antagonist in T Cells and/or NK Cells

Using standard cloning protocols know in the art, the coding sequence for the anti-TGFβRII single variable domain (such as SEQ ID NO: 72) with a secretion leader sequence will be cloned into lentiviral vectors that also encode CARs or TCRs of interest. The single variable domain sequence may be codon optimised using methods known in the art, in order to enhance expression in a mammalian system. Promoters used to drive the expression of the sequences will include constitutive and/or inducible promoters which are well known in the art.

Human PBMCs, or fractions thereof including T cells or NK cells, will be transduced by the recombinant lentiviral vectors described above such that immune cells are generated that co-express CAR/TCR and the single variable domain. Transfection or transduction methods are well known by a person skilled in the art.

### Example 5: Functional Assays

Assays can be used to demonstrate that the immune cells modified with the CAR/TCR and anti-TGFβRII single variable domain are resistant to inhibitory effects of TGFβ on effector functions following antigen recognition including cell proliferation, cytokine production and cytotoxicity. These experiments will use assays known in the art and include the supplementation of the culture medium with recombinant TGFβ and/or the use of tumour cell lines known to produce TGFβ, or transfected with TGFβ expression plasmids.

Using in vivo protocols known in the art such as murine xenograft models, it can be demonstrated that CAR/TCR transgenic immune cells modified to also express anti-TGFβRII single variable domain are superior to CAR/TCR modified cells alone at controlling tumour outgrowth, and extending survival. This can be achieved by comparing the results of such models with the results of administering recombinant anti-TGFβRII single variable domain alone at a range of doses by intraperitoneal (IP) or intravenous (IV) injection. These models will use tumours known to produce TGFβ or engineered to do so.

1. A pharmaceutical composition comprising:
(a) a TGF Receptor (TGFβR) antagonist which comprises a domain antibody; and
(b) a cell immunotherapy.

2. The pharmaceutical composition of claim 1, wherein the TGFβR antagonist binds to TGFβR or TGFβ.

3. The pharmaceutical composition of claim 1, wherein the TGFβR antagonist binds to TGF type II receptor (TGFβRII).

4. The pharmaceutical composition of claim 1, wherein the TGFβR antagonist is selected from: a domain antibody, a dual domain antibody, or a domain antibody attached to a single chain Fc region of an antibody.

5. The pharmaceutical composition of claim 1, wherein the cell immunotherapy is an immunomodulatory cell expressing a chimeric antigen receptor (CAR) or a modified T cell receptor (TCR).

6. The pharmaceutical composition of claim 5, wherein the CAR or TCR binds to a tumour associated antigen or pathogen antigen.

7. The pharmaceutical composition of claim 6, wherein the immunomodulatory cell is derived from an inflammatory T-lymphocyte, cytotoxic T-lymphocyte, regulatory T-lymphocyte, helper T-lymphocyte, natural killer cell, or a pluripotent stem cell from which lymphoid cells may be differentiated.

8. (canceled)

9. (canceled)

10. A polynucleotide comprising a sequence that encodes a TGFβ Receptor (TGFβR) antagonist which comprises a domain antibody, and a sequence that encodes a chimeric antigen receptor (CAR) or a T cell receptor (TCR).

11. The polynucleotide of claim 10, wherein the sequence that encodes a TGFβR antagonist and sequence that encodes a CAR or a TCR are each operably linked to a promoter element.

12. The polynucleotide of claim 10, wherein the CAR or TCR binds to a tumour associated antigen or pathogen antigen.

13. The polynucleotide of claim 10, wherein the TGFβR antagonist binds to TGFβR or TGFβ.

14. The polynucleotide of claim 10, wherein the TGFβR antagonist binds to TGF type II receptor (TGFβRII).

15. The polynucleotide of claim 10, wherein the TGFβR antagonist is selected from: a domain antibody, a dual domain antibody, or a domain antibody attached to a single chain Fc region of an antibody.

16. An expression vector comprising the polynucleotide of claim 10.

17. The expression vector of claim 16, which is a viral vector.

18. An immunomodulatory cell comprising the polynucleotide of claim 10.

19. The immunomodulatory cell of claim 18, which is derived from an inflammatory T-lymphocyte, cytotoxic T-lymphocyte, regulatory T-lymphocyte, helper T-lymphocyte, natural killer cell, or a pluripotent stem cell from which lymphoid cells may be differentiated.

20. (canceled)

21. (canceled)

22. A pharmaceutical composition, comprising:
(1) a plurality of immunomodulatory cells of claim 18; and
(2) a pharmaceutically acceptable carrier.

23. A method for producing an antigen-specific immunomodulatory cell, the method comprising introducing into an immunomodulatory cell the polynucleotide of claim 10.

24. A method of treatment comprising administering the expression vector of claim 16, to a subject.

25. A method of treatment comprising administering to a subject:
(a) a cell immunotherapy, and
(b) a TGF Receptor (TGFβR) antagonist which comprises a domain antibody.

26. The method of claim 25, wherein the cell immunotherapy is an immunomodulatory cell expressing a chimeric antigen receptor (CAR) or a modified T cell receptor (TCR).

27. A kit for the treatment of a disease selected from: cancer, an autoimmune disease or infection, comprising the pharmaceutical composition of claim 1.

28. A method of treatment comprising administering the immunomodulatory cell of claim 18 to a subject.


SEQUENCE LISTING (168 sequences)

SEQ ID NO: 1 - protein, 118 aa, Artificial Sequence
  DOM23h-33 amino acid
DMEVQLLESGGGLVQPGGSLRLSCAASGFTFSQYRMWWVRQAPGKGLEWVSAIAPSGDNTYYADSVKGRFTISRDNSKNTLYLQMNSLRAEDTAVYYCAKHRTSFDYWGQGTLVTVSS

SEQ ID NO: 2 - protein, 116 aa, Artificial Sequence
  DOM23h-251 amino acid
EVQLLESGGGLVQPGGSLRLSCAASGFTFSDYDMWWVRQAPGKGLEWVSKITQKGDFTYYADSVKGRFTISRDNSKNTLYLQMNSLRAEDTAVYYCAKDATHFDYWGQGTLVTVSS

SEQ ID NO: 3 - protein, 124 aa, Artificial Sequence
  DOM23h-262 amino acid
EVQLLESGGGLVQPGGSLRLSCAASGFTFFNYEMAWARQAPGKGLEWVSLISAEGTRTYYADSVKGRFTISRDNSKNTLYLQMNSLRAEDTAVYYCAKRRDASMGHTTRRFDYWGQGTLVTVSS

SEQ ID NO: 4 - protein, 124 aa, Artificial Sequence
  DOM23h-271 amino acid
EVQLLESGGGLVQPGGSLRLSCAASGFTFTEYRMWWVRQAPGKGLEWVSSIEPIGNRTYYADSVKGRFTISRDNSKNTLYLQMNSLRAEDTAVYYCAKQIPGHKWTANSRFDYWGQGTLVTVSS

SEQ ID NO: 5 - protein, 121 aa, Artificial Sequence
  DOM23h-348 amino acid
EVQLLESGGGLVQPGGSLRLSCAASGFTFSDYDMAWARQAPGKGLEWVSRISHSGYSTYYADSVKGRFTISRDNSKNTLYLQMNSLRAEDTAVYYCAKRSWDGVHAQFDYWGQGTLVTVSS

SEQ ID NO: 6 - protein, 116 aa, Artificial Sequence
  DOM23h-435 amino acid
EVQLLESGGGLVQPGGSLRLTCAASGFTFTDDRMWWVRQAPGKGLEWVSAIDPQGQHTYYADSVKGRFTISRDNSKNTLYLQMNSLRAEDTAVYYCAKHNSSFDYWGQGTLVTVSS

SEQ ID NO: 7 - protein, 116 aa, Artificial Sequence
  DOM23h-436 amino acid
EVQLLESGGGLVQPGGSLRLSCAASGFTFSDYKMGWVRQAPGKGLEWVSSIWPNGGLTYYADSVKGRFTISRDNSKNTLYLQMNSLRAEDTAVYYCAKDHMGFDYWGQGTLVTVSS

SEQ ID NO: 8 - protein, 116 aa, Artificial Sequence
  DOM23h-437 amino acid
EVQLLESGGGLVQPGGSLRLSCAASGFTFSDYRMWWVRQAPGKGLEWVSAIDSQGHTTYYADSVKGRFTISRDNSKNTLYLQMNSLRAEDTAVYYCAKYGGYFDYWGQGTLVTVSS

SEQ ID NO: 9 - protein, 119 aa, Artificial Sequence
  DOM23h-438 amino acid
EVQLLESGGGLVQPGGSLRLSCAASGFTFAQGDMWWVRQAPGKGLEWVSRIGMDGDKTYYADSVKGRFTISRDNSKNTLYLQMNSLRAEDTAVYYCAKGPSSTSPFDYWGQGTLVTVSS

SEQ ID NO: 10 - protein, 122 aa, Artificial Sequence
  DOM23h-439 amino acid
EVQLLESGGGLVQPGGSLRLSCAASGFTFGTEQMWWVRQAPGKGLEWVSRIDSPGGRTYYADSVKGRFTISRDNSKNTLYLQMNSLRAEDTAVYYCAKRHAAGVSGTYFDYWGQGTLVTVSS

SEQ ID NO: 11 - protein, 116 aa, Artificial Sequence
  DOM23h-440 amino acid
EVQLLESGGGLVQPGGSLRLSCAASGFTFTDDRMWWVRQAPGKGLEWVSAIDPQGQHTYYADSVKGRFTISRDNSKNTLYLQMNSLRAEDTAVYYCAKELLSFDYWGQGTLVTVSS

SEQ ID NO: 12 - protein, 124 aa, Artificial Sequence
  DOM23h-262-6 amino acid
EVQLLESGGGLVQPGGSLRLSCAASGFTFFNYEMAWARQAPGKGLEWVSLISAEGTRTYYANSVKGRFTISRDNSKNTLYLQMNSLRAEDTAVYYCAKRRDASMGHTTRRFDHWGQGTLVTVSS

SEQ ID NO: 13 - protein, 124 aa, Artificial Sequence
  DOM23h-262-10 amino acid
EVQLLESGGGLVQPGGSLRLSCAASGFTFFNYEMAWARRAPGKGLEWVSLISADGTRTYYANSVRGRFTISRDNSKNTLYLQMNSLRAEDTAVYYCAKRRDASMGHTTRRFDYSGQGTLVTVSS

SEQ ID NO: 14 - protein, 124 aa, Artificial Sequence
  DOM23h-271-3 amino acid
EVQLLESGGGLVQPGGSLRLSCTASGFTFTEYRMWWVRQAPGKGLEWVSLIEPIGNRTYYADSVKGRFTISRDNSKNTLYLQMNSLRAEDTAVYYCAKQIPGHMWTANPRSDYWGQGTQVTVSS

SEQ ID NO: 15 - protein, 124 aa, Artificial Sequence
  DOM23h-271-7 amino acid
EVQLLESGGGLVQPGGSLRLSCAASGFTFTEYRMWWVRQAPGKGLEWVSAIEPIGNRTYYADSVKGRFTISRDNSKNTLYLQMNSLRAEDTAVYYCAKQIPGRKWTANSRFDYWGQGTLVTVSS

SEQ ID NO: 16 - protein, 124 aa, Artificial Sequence
  DOM23h-271-12 amino acid
EVQLLESGGGLVQPGGSLRLSCTASGFTFTEYRMWWVRQAPGKGLEWVSLIEPIGNRTYYANSVRGRFTISRDNSKNTLYLQMNSLRAEDTAVYYCAKQIPGHMWTANPRSDYWGQGTQVTVSS

SEQ ID NO: 17 - protein, 124 aa, Artificial Sequence
  DOM23h-271-13 amino acid
EVQLLESGGGLVQPGGSLRLSCAASGFTFTEYRMWWVRQAPGKGLEWVSAIEPIGNRTYYANSVRGRFTISRDNSKNTLYLQMNSLRAEDTAVYYCAKQIPGRKWTANSRFDYWGQGTLVTVSS

SEQ ID NO: 18 - protein, 116 aa, Artificial Sequence
  DOM23h-437-4 amino acid
EVQLLESGGGLVQPGGSLRLSCAASGFTISDYRMWWVRQAPGKGLEWVSAIDSQGHTTYYADSVRGRFTISRDNSKNTLYLQMNSLRAEDTAVYYCAKYGGYFDYWGQGTLVTVSS

SEQ ID NO: 19 - protein, 116 aa, Artificial Sequence
  DOM23h-437-6 amino acid
EVQLLESGGGLVQPGGSLRLSCATSGFTFSDYRMWWVRQAPGKGLEWVSAIDSQGHTTYYADSVKGRFTISRDNSKNTLYLQMNSLRAEDTAVYYCAKYGGYFDYWGQGTLVTVSS

SEQ ID NO: 20 - protein, 116 aa, Artificial Sequence
  DOM23h-437-8 amino acid
EVQLLESGGGLVQPGGSLRLSCAASGFTISDYRMWWVRQAPGKGLEWVSAIDSQGHTTYYANSVRGRFTISRDNSKNTLYLQMNSLRAEDTAVYYCAKYGGYFDYWGQGTLVTVSS

SEQ ID NO: 21 - protein, 116 aa, Artificial Sequence
  DOM23h-437-9 amino acid
EVQLLESGGGLVQPGGSLRLSCATSGFTFSDYRMWWVRQAPGKGLEWVSAIDSQGHTTYYANSVRGRFTISRDNSKNTLYLQMNSLRAEDTAVYYCAKYGGYFDYWGQGTLVTVSS

SEQ ID NO: 22 - protein, 122 aa, Artificial Sequence
  DOM23h-439-6 amino acid
EVQLLESGGGLVRPGGSLRLSCAASGFTFGTEQMWWVRQAPGKGLEWVSRIDSPGGRTYYADSVRGRFTISRDNSKNTLYLQMNSLRAEDTAVYYCAKRHAAGVSGTYFDYWGQGTLVTVSS

SEQ ID NO: 23 - protein, 122 aa, Artificial Sequence
  DOM23h-439-8 amino acid
EVQLLESGGGLVRPGGSLRLSCAASGFTFGTEQMWWVRQAPGKGLEWVSRIDSPGGRTYYANSVRGRFTISRDNSKNTLYLQMNSLRAEDTAVYYCAKRHAAGVSGTYFDYWGQGTLVTVSS

SEQ ID NO: 24 - protein, 119 aa, Artificial Sequence
  DOM23h-802 amino acid
EVQLLESGGGLVQPGGSLRLSCAASGFTFSEGTMWWVRQAPGKGLEWVSAILAAGSNTYYADSVKGRFTISRDNSKNTLYLQMNSLRAEDTAVYYCAKKRQERDGFDYWGQGTLVTVSS

SEQ ID NO: 25 - protein, 119 aa, Artificial Sequence
  DOM23h-803 amino acid
EVQLLESGGGLVQPGGSLRLSCAASGFTFSAGRMWWVRQAPGKGLEWVSAINRDGTRTYYADSVKGRFTISRDNSKNTLYLQMNSLRAEDTAVYYCAKHDDGHGNFDYWGQGTLVTVSS

SEQ ID NO: 26 - protein, 120 aa, Artificial Sequence
  DOM23h-813 amino acid
EVQLLESGGGLVQPGGSLRLSCAASGSTFTDDRMWWVRQAPGKGLEWVSAIQPDGHTTYYADSVKGRFTISRDNSKNTLYLQMNSLRAEDTAVYYCAEQDVKGSSSFDYWGQGTLVTVSS

SEQ ID NO: 27 - protein, 119 aa, Artificial Sequence
  DOM23h-815 amino acid
EVQLLESGGGLVQPGGSLRLSCAASGFTFAEDRMWWVRQAPGKGLEWVSAIDPQGQHTYYADSVKGRFTISRDNSKNTLYLQMNSLRAEDTAVYYCAKQSTGSATSDYWGQGTLVTVSS

SEQ ID NO: 28 - protein, 124 aa, Artificial Sequence
  DOM23h-828 amino acid
EVQLLESGGGLVQPGGSLRLSCAASGFTFMSYRMWWVRQAPGKGLEWVSAISPSGSDTYYADSVKGRFTISRDNSKNTLYLQMNSLRAEDTAVYYCAKQVVEYSRTHKGVFDYWGQGTLVTVSS

SEQ ID NO: 29 - protein, 122 aa, Artificial Sequence
  DOM23h-830 amino acid
EVQLLESGGGLVQPGGFLRLSCAASGFTFEGYRMWWVRQAPGKGLEWVSAIDSLGDRTYYADSVKGRFTISRDNSKNTLYLQMNSLRAEDTAVYYCAKQGLTHQSPSTFDYWGQGTLVTVSS

SEQ ID NO: 30 - protein, 116 aa, Artificial Sequence
  DOM23h-831 amino acid
EVQLLESGGGLVQPGGSLRLSCAASGFTFEAYKMTWVRQAPGKGLEWVSYITPSGGQTYYADSVKGRFTISRDNSKNTLYLQMNSLRAEDTAVYYCAKYGSSFDYWGQGTLVTVSS

SEQ ID NO: 31 - protein, 119 aa, Artificial Sequence
  DOM23h-840 amino acid
EVQLLESGGGLVQPGGSLRLSCAASGFTFGDGRMWWVRQAPGKGLEWVSAIEGAGSDTYYADSVKGRFTISRDNSKNTLYLQMNSLRAEDTAVYYCAKQASRNSPFDYWGQGTLVTVSS

SEQ ID NO: 32 - protein, 120 aa, Artificial Sequence
  DOM23h-842 amino acid
EVQLLESGGGLVQPGGSLRLSCAASGFTFDDSEMAWARQAPGKGLEWVSLIRRNGNATYYADSVKGRFTISRDNSKNTLYLQMNSLRAEDTAVYYCAKVTKDRSVLFDYWGQGTLVTVSS

SEQ ID NO: 33 - protein, 120 aa, Artificial Sequence
  DOM23h-843 amino acid
EVQLLESGGGLVQPGGSLRLSCAASGFTFDQDRMWWVRQAPGKGLEWVSAIESGGHRTYYADSVKGRFTISRDNSKNTLYLQMNSLRAEDTAVYYCAKQNESGRSGFDYWGQGTLVTVSS

SEQ ID NO: 34 - protein, 119 aa, Artificial Sequence
  DOM23h-850 amino acid
EVQLLESGGGLVQPGGSLRLSCAASGFTFDAARMWWARQAPGKGLEWVSAIADIGNTTYYADSVKGRFTISRDNSKNTLYLQMNSLRAEDTAVYYCAKQSGSEDHFDYWGQGTLVTVSS

SEQ ID NO: 35 - protein, 121 aa, Artificial Sequence
  DOM23h-854 amino acid
EVQLLESGGGLVQPGGSLRLSCAASGFTFAQDRMWWVRQAPGKGLEWVSAISGSGGSTYYADSVKGRFTISRDNSKNTLYLQMNSLRAEDTAVYYCAKQDLHGTSSLFDYWGQGTLVTVSS

SEQ ID NO: 36 - protein, 120 aa, Artificial Sequence
  DOM23h-855 amino acid
EVQLLESGGGLVQPGGSLRLSCAASGFTFENTSMGWVRQAPGKGLEWVSRIDPKGSHTYYADSVKGRFTISRDNSKNTLYLQMNSLRAEDTAVYYCAKQRELGKSHFDYWGQGTLVTVSS

SEQ ID NO: 37 - protein, 119 aa, Artificial Sequence
  DOM23h-865 amino acid
EVQLLESGGGLVQPGGSLRLSCAASGFTFRSYEMTWVRQAPGKGLEWVSKIDPSGRFTYYADSVKGRFTISRDNSKNTLYLQMNSLRAEDTAVYYCAKGRTDLQLFDYWGQGTLVTVSS

SEQ ID NO: 38 - protein, 121 aa, Artificial Sequence
  DOM23h-866 amino acid
EVQLLESGGGLVQPGGSLRLSCAASGFTFSNYWMRWARQAPGKGLEWVSYITPKGDHTYYADSVKGRFTISRDNSKNTLYLQMNSLRAEDTAVYYCAESLHNERVKHFDYWGQGTLVTVSS

SEQ ID NO: 39 - protein, 121 aa, Artificial Sequence
  DOM23h-874 amino acid
EVQLLESGGGLVQPGGSLRLSCAASGFTFTSYRMWWVRQAPGKGLEWVSVIDSTGSATYYADSVKGRFTISRDNSKNTLYLQMNSLRAEDTAVYYCAKQQAGSAMGEFDYWGQGTLVTVSS

SEQ ID NO: 40 - protein, 116 aa, Artificial Sequence
  DOM23h-883 amino acid
EVQLLESGGGLVQPGGSLRLSCAASGFTFVNYRMWWVRQAPGKGLEWVSAISGSGDKTYYADSVKGRFTISRDNSKNTLYLQMNSLRAEDTAVYYCAKHGLSFDYWGQGTLVTVSS

SEQ ID NO: 41 - protein, 116 aa, Artificial Sequence
  DOM23h-903 amino acid
EVQLLESGGGLVQPGGSLRLSCAASGFTFNDMRMWWVRQAPGKGLEWVSVINADGNRTYYADSVKGRFTISRDNSKNTLYLQMNSLRAEDTAVYYCAKDGLPFDYWGQGTLVTVSS

SEQ ID NO: 42 - protein, 123 aa, Artificial Sequence
  DOM23m-4 amino acid
EVQLLESGGGLVQPGGSLRLSCAASGFTFTTYGMGWVRQAPGKGLEWVSWIEKTGNKTYYADSVKGRFTISRDNSKNTLYLQMNSLRAEDTAVYYCAKAGRHIKVRSRDFDYWGQGTLVTVSS

SEQ ID NO: 43 - protein, 123 aa, Artificial Sequence
  DOM23m-29 amino acid
EVQLLESGGGLVQPGGSLRLSCAASGFTFKRYSMGWVRQAPGKGLEWVSVINDLGSLTYYADSVKGRFTISRDNSKNTLYLQMNSLRAEDTAVYYCAKGNISMVRPGSWFDYWGQGTLVTVSS

SEQ ID NO: 44 - protein, 123 aa, Artificial Sequence
  DOM23m-32 amino acid
EVQLLESGGGLVQPGGSLRLSCAASGFTFFEYPMGWVRQAPGKGLEWVSVISGDGQRTYYADSVKGRFTISRDNSKNTLYLQMNSLRAEDTAVYYCAKSHTGTVRHLETFDYWGQGTLVTVSS

SEQ ID NO: 45 - protein, 120 aa, Artificial Sequence
  DOM23m-62 amino acid
EVQLLESGGGLVQPGGSLRLSCAASGFTFGQESMYWVRQAPGKGLEWVSAISGSGGSTYYADSVKGRFTISRDNSKNTLYLQMNSLRAEDTAVYYCAKSGTRIKQGFDYWGQGTLVTVSS

SEQ ID NO: 46 - protein, 123 aa, Artificial Sequence
  DOM23m-71 amino acid
EVQLLESGGGLVQPGGSLRLSCAASGFTFMDYRMYWVRQAPGKGLEWVSGIDPTGLRTYYADSVKGRFTISRDNSKNTLYLQMNSLRAEDTAVYYCAKIKWGEMGSYKTFDYWGQGTLVTVSS

SEQ ID NO: 47 - protein, 124 aa, Artificial Sequence
  DOM23m-72 amino acid
EVQLLESGGGLVQPGGSLRLSCAASGFTFMDYDMSWVRQAPGKGLEWVSMIREDGGKTYYADSVKGRFTISRDNSKNTLYLQMNSLRAEDTAVYYCAKARVPYRRGHRDNFDYWGQGTLVTVSS

SEQ ID NO: 48 - protein, 120 aa, Artificial Sequence
  DOM23m-81 amino acid
EVQLLESGGGLVQPGGSLRLSCAASGFTFEPVIMGWVRQAPGKGLEWVSAIEARGGGTYYADSVKGRFTISRDNSKNTLYLQMNSLRAEDTAVYYCAKPGRHLSQDFDYWGQGTLVTVSS

SEQ ID NO: 49 - protein, 119 aa, Artificial Sequence
  DOM23m-99 amino acid
EVQLLESGGGLVQPGGSLRLSCAASGFTFDRYRMMWVRQAPGKGLEWVSTIDPAGMLTYYADSVKGRFTISRDNSKNTLYLQMNSLRAEDTAVYYCAKRLASRSHFDYWGQGTLVTVSS

SEQ ID NO: 50 - protein, 119 aa, Artificial Sequence
  DOM23m-101 amino acid
EVQLLESGGGLVQPGGSLRLSCAASGFTFSEYDMAWVRQAPGKGLEWVSRIRSDGVRTYYADSVKGRFTISRDNSKNTLYLQMNSLRAEDTAVYYCAKDRAKNGWFDYWGQGTLVTVSS

SEQ ID NO: 51 - protein, 119 aa, Artificial Sequence
  DOM23m-352 amino acid
EVQLLESGGGLVQPGGSLRLSCAASGFTFDKYKMAWVRQAPGKGLEWVSLIFPNGVPTYYANSVKGRFTISRDNSKNTLYLQMNSLRAEDTAVYYCAKYSGQGRDFDYWGQGTLVTVSS

SEQ ID NO: 52 - protein, 124 aa, Artificial Sequence
  DOM23h-271-21 amino acid
EVQLLESGGGLVQPGGSLRLSCAASGFTFTEYRMWWVRQAPGKGLEWVSAIEPIGNRTYYADSVKGRFTISRDNSKNTLYLQMNSLRAEDTAVYYCAKQMPGRKWTAKFRWDYWGQGTLVIVSS

SEQ ID NO: 53 - protein, 124 aa, Artificial Sequence
  DOM23h-271-22 amino acid
EVQLLESGGGLVQPGGSLRLSCAASGFTFTEYRMWWVRQAPGKGLEWVSAIEPIGNRTYYADSVKGRFTISRDNSKNTLYLQMNSLRAEDTAVYYCAKQMPGQKWMAKSRFDYWGQGTLVTVSS

SEQ ID NO: 54 - protein, 124 aa, Artificial Sequence
  DOM23h-271-27 amino acid
EVQLLESGGGLVQPGGSLRLSCAASGFTFTEYRMWWVRQAPGKGLEWVSAIEPIGQKTYYADSVKGRFTISRDNSKNTLYLQMNSLRAEDTAVYYCAKQIPGRKWTANSRFDYWGQGTLVIVSS

SEQ ID NO: 55 - protein, 124 aa, Artificial Sequence
  DOM23h-271-101 amino acid
EVQLLESGGGLVQPGGSLRLSCAASGFTFTEYRMWWVRQAPGKGLEWVSAIEPIGNRTYYADSVKGRFTISRDNSKNTLYLQMNSLRAEDTAVYYCAKQIPGRKWTANGRKDYWGQGTLVTVSS

SEQ ID NO: 56 - protein, 124 aa, Artificial Sequence
  DOM23h-271-102 amino acid
EVQLLESGGGLVQPGGSLRLSCAASGSTFTEYRMWWVRQAPGKGLEWVSAIEPIGHRTYYADSVKGRFTISRDNSKNTLYLQMNSLRAEDTAVYYCAKQIPGRKWTANSRFDYWGQGTLVTVSS

SEQ ID NO: 57 - protein, 124 aa, Artificial Sequence
  DOM23h-271-105 amino acid
EVQLLESGGGLVQPGGSLRLSCAASGFTFTEYRMWWVRQAPGKGLEWVSAIEPIGNRTYYADSVKGRFTISRDNSKNTLYLQMNSLRAEDTAVYYCAKQIPGQRWTGNSRFDYWGQGTLVTVSS

SEQ ID NO: 58 - protein, 124 aa, qArtificial Sequence
  DOM23h-271-106 amino acid
EVQLLESGGGLVQPGGSLRLSCAASGFTFTEYRMWWVRQAPGKGLEWVSAIEPIGNRTYYADSVKGRFTISRDNSKNTLYLQMNSLRAEDTAVYYCAKQFPGRKWTANSRSDYWGQGTLVTVSS

SEQ ID NO: 59 - protein, 124 aa, Artificial Sequence
  DOM23h-271-114 amino acid
EVQLLESGGGLVQPGGSLRLSCAASGFTFTEYRMWWVRQAPGKGLEWVSAIEPIGNRTYYADSVKGRFTISRDNSKNTLYLQMNSLRAEDTAVYYCAKQIPGRKGTANSRFDYWGQGTLVTVSS

SEQ ID NO: 60 - protein, 124 aa, Artificial Sequence
  DOM23h-271-39 amino acid
EVQLLESGGGLVQPGGSLRLSCAASGFTFTEYRMWWVRQAPGKGLEWVSAIEPIGNRTYYARSVDGRFTISRDNSKNTLYLQMNSLRAEDTAVYYCAKQMPGQKWMAKSRFDYWGQGTLVTVSS

SEQ ID NO: 61 - protein, 124 aa, Artificial Sequence
  DOM23h-271-40 amino acid
EVQLLESGGGLVQPGGSLRLSCAASGFTFTEYRMWWVRQAPGKGLEWVSAIEPIGNRTYYARSVFGRFTISRDNSKNTLYLQMNSLRAEDTAVYYCAKQMPGQKWMAKSRFDYWGQGTLVTVSS

SEQ ID NO: 62 - protein, 120 aa, Artificial Sequence
  DOM23h-855-21 amino acid
EVQLLESGGGLVQPGGSLRLSCAASGFTFENTSMGWVRQAPGKGLEWVSRIDPKGSHTYYTDSVKGRFTISRDNSKNTLYLQMNSLRAEDTAVYYCAKQRELGKSYFDYWGQGTLVTVSS

SEQ ID NO: 63 - protein, 120 aa, Artificial Sequence
  DOM23h-843-13 amino acid
EVQLLESGGGLVQPGGSLRLSCAASGFTFDQDRMWWVRQAPGKGLEWVSAIESGGHRTYYADSVKGRFTISRDNSKNTLYLQMNSLRAEDTAVYYCANQNKSGRSGFDYWGQGTLVTVSS

SEQ ID NO: 64 - protein, 122 aa, Artificial Sequence
  DOM23h-439-20 amino acid
EVQLLESGGGLVQPGGSLRLSCAASGFTFGTEQMWWVRQAPGKGLEFVSRIDSPGGRTYYADSVKGRFTISRDNSKNTLYLQMNSLRAEDTAVYYCAKRHAAGVSGTYFDYWGQGTLVTVSS

SEQ ID NO: 65 - protein, 124 aa, Artificial Sequence
  DOM23h-271-50 amino acid
EVQLLESGGGLVQPGGSLRLSCAASGSTFTEYRMWWVRQAPGKGLEWVSAIEPIGHRTYYADSVKGRFTISRDNSKNTLYLQMNSLRAEDTAVYYCAKQAPGEKWLARGRLDYWGQGTLVTVSS

SEQ ID NO: 66 - protein, 122 aa, Artificial Sequence
  DOM23h-439-25 amino acid
EVQLLESGGGLVQPGGSLRLSCAASGFTFGTEQMWWVRQAPGKGLEFVSRIDSPGGRTYYADSVKGRFTISRDNSKNTLYLQMNSLRAEDTAVYYCAKRRPTGVSGTFYDYWGQGTLVTVSS

SEQ ID NO: 67 - protein, 124 aa, Artificial Sequence
  DOM23h-271-123 amino acid
EVQLLESGGGLVQPGGSLRLSCAASGSTFTEYRMWWVRQAPGKGLEWVSAIEPIGHRTYYADSVKGRFTISRDNSKNTLYLQMNSLRAEDTAVYYCAKQAPGEKWARRWDLDYWGQGTLVTVSS

SEQ ID NO: 68 - protein, 122 aa, Artificial Sequence
  DOM23h-439-35 amino acid
EVQLLESGGGLVQPGGSLRLSCAASGFTFGTDQMWWVRQAPGKGLEFVSRIDSPGGRTYYANSVKGRFTISRDNSKNTLYLQMNSLRAEDTAVYYCAKRQPAGVSGKYVDYWGQGTLVTVSS

SEQ ID NO: 69 - protein, 124 aa, Artificial Sequence
  DOM23h-271-129 amino acid
EVQLLESGGGLVQPGGSLRLSCAASGSTFTEYRMWWVRQAPGKGLEWVSAIEPIGHRTYYADSVKGRFTISRDNSKNTLYLQMNSLRAEDTAVYYCAKQAPNQRYVARGRLDYWGQGTLVTVSS

SEQ ID NO: 70 - protein, 123 aa, Artificial Sequence
  DOM23h-439-40 amino acid
EVQLLESGGGLVQPGGSLRLSCAASGFTFGTEQMWWVRQAPGKGLEFVSRIDSPGGRTYYADSVKGRFTISRDNSKNTLYLQMNSLRAEDTAVYYCAKRRPTGVSGTFYDYWGQGTLVTVSSA

SEQ ID NO: 71 - protein, 123 aa, Artificial Sequence
  DOM23h-439-41 amino acid
EVQLLESGGGLVQPGGSLRLSCAASGFTFGTEQMWWVRQAPGKGLEFISRIDSPGGRTYYADSVKGRFTISRDNSKNTLYLQMNSLRAEDTAVYYCAKRRPTGVSGTFYDYWGQGTLVTVSSA

SEQ ID NO: 72 - protein, 123 aa, Artificial Sequence
  DOM23h-439-42 amino acid
EVQLLESGGGLVQPGGSLRLSCAASGFTFGTEQMWWVRQAPGKGLEFVSRIDSPGGRTYYANSVKGRFTISRDNSKNTLYLQMNSLRAEDTAVYYCAKRRPTGVSGTFYDYWGQGTLVTVSSA

SEQ ID NO: 73 - protein, 123 aa, Artificial Sequence
  DOM23h-439-43 amino acid
EVQLLESGGGLVQPGGSLRLSCAASGFTFGTEQMWWVRQAPGKGLEFVSRIDSPGGRTYYADSVRGRFTISRDNSKNTLYLQMNSLRAEDTAVYYCAKRRPTGVSGTFYDYWGQGTLVTVSSA

SEQ ID NO: 74 - protein, 123 aa, Artificial Sequence
  DOM23h-439-44 amino acid
EVQLLESGGGLVQPGGSLRLSCAASGFTFGTEQMWWVRQAPGKGLEFVSRIDSPGGRTYYANSVRGRFTISRDNSKNTLYLQMNSLRAEDTAVYYCAKRRPTGVSGTFYDYWGQGTLVTVSSA

SEQ ID NO: 75 - protein, 125 aa, Artificial Sequence
  DOM23h-271-130 amino acid
EVQLLESGGGLVQPGGSLRLSCAASGSTFTEYRMWWVRQAPGKGLEWVSAIEPIGHRTYYADSVKGRFTISRDNSKNTLYLQMNSLRAEDTAVYYCAKQAPGEKWARRWDLDYWGQGTLVTVSSA

SEQ ID NO: 76 - protein, 125 aa, Artificial Sequence
  DOM23h-271-131 amino acid
EVQLLESGGGLVQPGGSLRLSCAASGSTFTEYRMWWVRQAPGKGLEWVSAIEPIGHRTYYANSVKGRFTISRDNSKNTLYLQMNSLRAEDTAVYYCAKQAPGEKWARRWDLDYWGQGTLVTVSSA

SEQ ID NO: 77 - protein, 125 aa, Artificial Sequence
  DOM23h-271-132 amino acid
EVQLLESGGGLVQPGGSLRLSCAASGSTFTEYRMWWVRQAPGKGLEWVSAIEPIGHRTYYADSVRGRFTISRDNSKNTLYLQMNSLRAEDTAVYYCAKQAPGEKWARRWDLDYWGQGTLVTVSSA

SEQ ID NO: 78 - protein, 125 aa, Artificial Sequence
  DOM23h-271-133 amino acid
EVQLLESGGGLVQPGGSLRLSCAASGSTFTEYRMWWVRQAPGKGLEWVSAIEPIGHRTYYANSVRGRFTISRDNSKNTLYLQMNSLRAEDTAVYYCAKQAPGEKWARRWDLDYWGQGTLVTVSSA

SEQ ID NO: 79 - protein, 125 aa, Artificial Sequence
  DOM23h-271-134 amino acid
EVQLLESGGGLVQPGGSLRLSCAASGSTFTEYRMWWVRQAPGKGLEWISAIEPIGHRTYYADSVKGRFTISRDNSKNTLYLQMNSLRAEDTAVYYCAKQAPGEKWARRWDLDYWGQGTLVTVSSA

SEQ ID NO: 80 - protein, 125 aa, Artificial Sequence
  DOM23h-271-135 amino acid
EVQLLESGGGLVQPGGSLRLSCAASGSTFTEYRMWWVRQAPGKGLEWVSAIEPIGHRTYYADSVKGRFTISRDNSKNTLYLQMNSLRAEDTAVYYCAKQAPNQRYVARGRLDYWGQGTLVTVSSA

SEQ ID NO: 81 - protein, 125 aa, Artificial Sequence
  DOM23h-271-136 amino acid
EVQLLESGGGLVQPGGSLRLSCAASGSTFTEYRMWWVRQAPGKGLEWVSAIEPIGHRTYYANSVKGRFTISRDNSKNTLYLQMNSLRAEDTAVYYCAKQAPNQRYVARGRLDYWGQGTLVTVSSA

SEQ ID NO: 82 - protein, 125 aa, Artificial Sequence
  DOM23h-271-137 amino acid
EVQLLESGGGLVQPGGSLRLSCAASGSTFTEYRMWWVRQAPGKGLEWVSAIEPIGHRTYYANSVRGRFTISRDNSKNTLYLQMNSLRAEDTAVYYCAKQAPNQRYVARGRLDYWGQGTLVTVSSA

SEQ ID NO: 83 - protein, 123 aa, Artificial Sequence
  DOM23h-439-47 amino acid
EVQLLESGGGLVQPGGSLRLSCAASGFTFGTEQMWWVRQAPGKGLEFISRIDSPGGRTYYANSVKGRFTISRDNSKNTLYLQMNSLRAEDTAVYYCAKRRPTGVSGTFYDYWGQGTLVTVSSA

SEQ ID NO: 84 - protein, 123 aa, Artificial Sequence
  DOM23h-439-48 amino acid
EVQLLESGGGLVQPGGSLRLSCAASGFTFGTEQMWWVRQAPGKGLEFISRIDSPGGRTYYANSVRGRFTISRDNSKNTLYLQMNSLRAEDTAVYYCAKRRPTGVSGTFYDYWGQGTLVTVSSA

SEQ ID NO: 85 - DNA, 348 bp, Artificial Sequence
  DOM23h-33 nucleic acid
gaggtgcagctgttggagtctgggggaggcttggtacagcctggggggtccctgcgtctctcctgtgcagcctccggattcaccttttcgcagtatcggatgtggtgggtccgccaggctccagggaagggtctagagtgggtctcagcgattgcgccttctggtgataatacatactacgcagactccgtgaagggccggttcaccatctcccgcgacaattccaagaacacgctgtatctgcaaatgaacagcctgcgtgccgaggacaccgcggtatattactgtgcgaaacatcggacttcgtttgactactggggtcagggaaccctggtcaccgtctcgagc

SEQ ID NO: 86 - DNA, 348 bp, Artificial Sequence
  DOM23h-251 nucleic acid
gaggtgcagctgttggagtctgggggaggcttggtacagcctggggggtccctgcgtctctcctgtgcagcctccggattcaccttttcggattatgatatgtggtgggtccgccaggctccagggaagggtctggagtgggtctcaaagattacgcagaagggtgattttacatactacgcagactccgtgaagggccggttcaccatctcccgcgacaattccaagaacacgctgtatctgcaaatgaacagcctgcgtgccgaggacaccgcggtatattactgtgcgaaagatgctactcattttgactactggggtcagggaaccctggtcaccgtctcgagc

SEQ ID NO: 87 - DNA, 372 bp, Artificial Sequence
  DOM23h-262 nucleic acid
gaggtgcagctgttggagtctgggggaggcttggtacagcctggggggtccctgcgtctctcctgtgcagcctccggattcaccttttttaattatgagatggcgtgggcccgccaggctccagggaagggtctagagtgggtctcattgattagtgctgagggtacgaggacatactacgcagactccgtgaagggccggttcaccatctcccgcgacaattccaagaacacgctgtatctgcaaatgaacagcctgcgtgccgaggacaccgcggtatattactgtgcaaaacggcgggatgctagtatgggtcatactactcggcggtttgactactggggtcagggaaccctggtcaccgtctcgagc

SEQ ID NO: 88 - DNA, 373 bp, Artificial Sequence
  DOM23h-271 nucleic acid
gaggtgcagctgttggagtctgggggaggcttggtacagcctggggggtccctgcgtctctcctgtgcagcctccggattcacctttacggagtataggatgtggtgggtccgccaggctccagggaagggtctagagtgggtctcatcgattgagccgattggtaatcgtacatactacgcagactccgtgaagggccggttcaccatctcccgcgacaattccaagaacacgctgtatctgcaaatgaacagcctgcgtgccgaggataccgcggtatattactgtgcgaaacagattccggggcataagtggactgctaattcgcggtttgactactggggtcagggaaccctggtcaccgtctcgagcg

SEQ ID NO: 89 - DNA, 363 bp, Artificial Sequence
  DOM23h-348 nucleic acid
gaggtgcagctgttggagtctggggggggcttggtacagcctggggggtccctgcgtctctcctgtgcagcctccggattcacctttagtgattatgatatggcgtgggcccgccaggctccagggaagggtctagagtgggtctcacgtatttctcatagtggttattctacatactacgcagactccgtgaagggccggttcaccatctcccgcgacaattccaagaacacgctgtatctgcaaatgaacagcctgcgtgccgaggacaccgcggtatattactgtgcgaaacgttcgtgggatggggttcatgcgcagtttgactactggggtcagggaaccctggtcaccgtctcgagc

SEQ ID NO: 90 - DNA, 349 bp, Artificial Sequence
  DOM23h-435 nucleic acid
gaggtgcagctgttggagtctgggggaggcttggtacagcctggggggtccctgcgtctcacctgtgcagcctccggattcacctttacggatgataggatgtggtgggtccgccaggctccagggaagggtctagagtgggtctcagctattgatcctcagggtcagcatacatactacgcagactccgtgaagggccggttcaccatctcccgcgacaattccaagaacacgctgtatctgcaaatgaacagcctgcgtgccgaggataccgcggtatattactgtgcgaaacataattcgagttttgactactggggtcagggaaccctggtcaccgtctcgagcg

SEQ ID NO: 91 - DNA, 349 bp, Artificial Sequence
  DOM23h-436 nucleic acid
gaggtgcagctgttggagtctgggggaggcttggtacagcctggggggtccctgcgtctctcctgtgcagcctccggattcacctttagtgattataagatgggttgggtccgccaggctccagggaagggtctagagtgggtctcaagtatttggcctaatggtggtttgacatactacgcagactccgtgaagggccggttcaccatctcccgcgacaattccaagaacacgctgtatctgcaaatgaacagcctgcgtgccgaggataccgcggtatattactgtgcgaaagatcatatgggttttgactactggggtcagggaaccctggtcaccgtctcgagcg

SEQ ID NO: 92 - DNA, 349 bp, Artificial Sequence
  DOM23h-437 nucleic acid
gaggtgcagctgttggagtctgggggaggcttggtacagcctggggggtccctgcgtctctcctgtgcagcctccggattcaccttttctgattatcgtatgtggtgggtccgccaggctccagggaagggtctagagtgggtctcagcgattgattctcagggtcatacgacatactacgcagactccgtgaagggccggttcaccatctcccgcgacaattccaagaacacgctgtatctgcaaatgaacagcctgcgtgccgaggataccgcggtatattactgtgcgaaatatgggggttattttgactactggggtcagggaaccctggtcaccgtctcgagcg

SEQ ID NO: 93 - DNA, 358 bp, Artificial Sequence
  DOM23h-438 nucleic acid
gaggtgcagctgttggagtctgggggaggcttggtacagcctggggggtccctgcgtctctcctgtgcagcctccggattcacctttgcgcagggggatatgtggtgggtccgccaggctccagggaagggtctggagtgggtctcacgtattggtatggatggtgataagacatactacgcagactccgtgaagggccggttcaccatctcccgcgacaattccaagaacacgctgtatctgcaaatgaacagcctgcgtgccgaggacaccgcggtatattactgtgcgaaaggtccttcgagtactagtccgtttgactactggggtcagggaaccctggtcaccgtctcgagcg

SEQ ID NO: 94 - DNA, 367 bp, Artificial Sequence
  DOM23h-439 nucleic acid
gaggtgcagctgttggagtctgggggaggcttggtacagcctggggggtccctgcgtctctcctgtgcagcctccggattcacctttgggacggagcagatgtggtgggtccgccaggctccagggaagggtctagagtgggtctcacgtattgattcgcctggtgggaggacatactacgcagactccgtgaagggccggttcaccatctcccgcgacaattccaagaacacgctgtatctgcaaatgaacagcctgcgtgccgaggacaccgcggtatattactgtgcgaaacggcatgcggctggggtttcgggtacttattttgactactggggtcagggaaccctggtcaccgtctcgagcg

SEQ ID NO: 95 - DNA, 349 bp, Artificial Sequence
  DOM23h-440 nucleic acid
gaggtgcagctgttggagtctgggggaggcttggtacagcctggggggtccctgcgtctctcctgtgcagcctccggattcacctttacggatgataggatgtggtgggtccgccaggctccagggaagggtctagagtgggtctcagctattgatcctcagggtcagcatacatactacgcagactccgtgaagggccggttcaccatctcccgcgacaattccaagaacacgctgtatctgcaaatgaacagcctgcgtgccgaggacaccgcggtatattactgtgcgaaagagctgcttagttttgactactggggtcagggaaccctggtcaccgtctcgagcg

SEQ ID NO: 96 - DNA, 372 bp, Artificial Sequence
  DOM23h-262-6 nucleic acid
gaggtgcagctgttggagtctgggggaggcttggtacagcctggggggtccctgcgtctctcctgtgcagcctccggattcaccttttttaattatgagatggcgtgggcccgccaggctccagggaagggcctagagtgggtctcattgattagtgctgagggtacgaggacatactacgcaaactccgtgaagggccggttcaccatctcccgcgacaattccaagaacacgctgtatctgcaaatgaacagcctgcgtgccgaggacaccgcggtatattactgtgcaaaacggcgggatgctagtatgggtcatactactcggcggtttgaccactggggtcagggaaccctggtcaccgtctcgagc

SEQ ID NO: 97 - DNA, 371 bp, Artificial Sequence
  DOM23h-262-10 nucleic acid
gaggtgcagctgttggagtctgggggaggcttagtacagcccggggggtccctgcgtctctcctgtgcagcctccggattcaccttttttaattatgagatggcgtgggcccgccgggctccagggaagggtctagagtgggtctcattgattagtgctgatggtacgaggacatactacgcaaactccgtgaggggccggttcaccatctcccgcgacaattccaagaaccgctgtatctgcaaatgaacagcctgcgtgccgaggacaccgcggtatattactgtgcaaaacggcgggatgctagtatgggtcatactactcggcggtttgactactcgggtcagggaaccctggtcaccgtctcgagc

SEQ ID NO: 98 - DNA, 372 bp, Artificial Sequence
  DOM23h-271-3 nucleic acid
gaggtgcagctgttggagtctgggggaggcttggtacagcctggggggtccctgcgtctctcctgcacagcctccggattcacctttacggagtataggatgtggtgggtccgccaggctccagggaagggtctagagtgggtctcattgattgagccgattggtaatcgtacatactacgcagactccgtgaagggccggttcaccatctcccgcgacaattccaagaacacgctgtatctgcaaatgaacagcctgcgtgctgaggataccgcggtatattactgtgcgaaacagattccggggcatatgtggactgctaatccgcggtctgactactggggtcagggaacccaggtcaccgtctcgagc

SEQ ID NO: 99 - DNA, 372 bp, Artificial Sequence
  DOM23h-271-7 nucleic acid
gaggtgcagctgttggagtctgggggaggcttggtacagcctggggggtccctgcgtctctcctgtgcagcctccggattcacctttacggagtataggatgtggtgggtccgccaggctccggggaagggtctcgagtgggtctcagcgattgagccgattggtaatcgtacatactacgcagactccgtgaagggccggttcaccatctcccgcgacaattccaagaacacgctgtatctgcaaatgaacagcctgcgtgccgaggataccgcggtatattactgtgcgaaacagattccggggcgtaagtggactgctaattcgcggtttgactactggggtcagggaaccctggtcaccgtctcgagc

SEQ ID NO: 100 - DNA, 372 bp, Artificial Sequence
  DOM23h-271-12 nucleic acid
gaggtgcagctgttggagtctgggggaggcttggtacagcctggggggtccctgcgtctctcctgcacagcctccggattcacctttacggagtataggatgtggtgggtccgccaggctccagggaagggtctagagtgggtctcattgattgagccgattggtaatcgtacatactacgcaaactccgtgaggggccggttcaccatctcccgcgacaattccaagaacacgctgtatctgcaaatgaacagcctgcgtgctgaggataccgcggtatattactgtgcgaaacagattccggggcatatgtggactgctaatccgcggtctgactactggggtcagggaacccaggtcaccgtctcgagc

SEQ ID NO: 101 - DNA, 372 bp, Artificial Sequence
  DOM23h-271-13 nucleic acid
gaggtgcagctgttggagtctgggggaggcttggtacagcctggggggtccctgcgtctctcctgtgcagcctccggattcacctttacggagtataggatgtggtgggtccgccaggctccggggaagggtctcgagtgggtctcagcgattgagccgattggtaatcgtacatactacgcaaactccgtgaggggccggttcaccatctcccgcgacaattccaagaacacgctgtatctgcaaatgaacagcctgcgtgccgaggataccgcggtatattactgtgcgaaacagattccggggcgtaagtggactgctaattcgcggtttgactactggggtcagggaaccctggtcaccgtctcgagc

SEQ ID NO: 102 - DNA, 348 bp, Artificial Sequence
  DOM23h-437-4 nucleic acid
gaggtgcagctgttggagtctgggggaggcttggtacagcctggggggtccctgcgtctctcctgtgcagcctccggattcaccatttctgattatcgtatgtggtgggtccgccaggctccagggaagggtctagagtgggtctcagcgattgattctcagggtcatacgacatactacgcagactccgtgaggggccggttcaccatctcccgcgacaattccaagaacacgctgtatctgcaaatgaacagcctgcgtgccgaggataccgcggtatattactgtgcgaaatatgggggttattttgactactggggtcagggaaccctggtcaccgtctcgagc

SEQ ID NO: 103 - DNA, 348 bp, Artificial Sequence
  DOM23h-437-6 nucleic acid
gaggtgcagctgttggagtctgggggaggcttggtacagcctggggggtccctgcgtctctcctgtgcaacctccggattcaccttttctgattatcgtatgtggtgggtccgccaggctccagggaagggtctagagtgggtctcagcgattgattctcagggtcatacgacatactacgcagactccgtgaagggccggttcaccatctcccgcgacaattccaagaacacgctgtatctgcaaatgaacagcctgcgcgccgaggataccgcggtatattactgtgcgaaatatgggggttattttgactactggggtcagggaaccctggtcaccgtctcgagc

SEQ ID NO: 104 - DNA, 348 bp, Artificial Sequence
  DOM23h-437-8 nucleic acid
gaggtgcagctgttggagtctgggggaggcttggtacagcctggggggtccctgcgtctctcctgtgcagcctccggattcaccatttctgattatcgtatgtggtgggtccgccaggctccagggaagggtctagagtgggtctcagcgattgattctcagggtcatacgacatactacgcaaactccgtgaggggccggttcaccatctcccgcgacaattccaagaacacgctgtatctgcaaatgaacagcctgcgtgccgaggataccgcggtatattactgtgcgaaatatgggggttattttgactactggggtcagggaaccctggtcaccgtctcgagc

SEQ ID NO: 105 - DNA, 348 bp, Artificial Sequence
  DOM23h-437-9 nucleic acid
gaggtgcagctgttggagtctgggggaggcttggtacagcctggggggtccctgcgtctctcctgtgcaacctccggattcaccttttctgattatcgtatgtggtgggtccgccaggctccagggaagggtctagagtgggtctcagcgattgattctcagggtcatacgacatactacgcaaactccgtgaggggccggttcaccatctcccgcgacaattccaagaacacgctgtatctgcaaatgaacagcctgcgcgccgaggataccgcggtatattactgtgcgaaatatgggggttattttgactactggggtcagggaaccctggtcaccgtctcgagc

SEQ ID NO: 106 - DNA, 366 bp, Artificial Sequence
  DOM23h-439-6 nucleic acid
gaggtgcagctgttggagtctgggggaggcttggtacggcctggggggtccctgcgtctctcctgtgcagcctccggattcacctttgggacggagcagatgtggtgggtccgccaggctccagggaagggtctagagtgggtctcacgaattgattcacctggtgggaggacatactacgcagactccgtgaggggccggttcaccatctcccgcgacaattccaagaacacgctgtatctgcaaatgaacagcctgcgtgccgaggacaccgcggtatattactgtgcgaaacggcatgcggctggggtttcgggtacttattttgactactggggtcagggaaccctggtcaccgtctcgagc

SEQ ID NO: 107 - DNA, 366 bp, Artificial Sequence
  DOM23h-439-8 nucleic acid
gaggtgcagctgttggagtctgggggaggcttggtacggcctggggggtccctgcgtctctcctgtgcagcctccggattcacctttgggacggagcagatgtggtgggtccgccaggctccagggaagggtctagagtgggtctcacgaattgattcacctggtgggaggacatactacgcaaactccgtgaggggccggttcaccatctcccgcgacaattccaagaacacgctgtatctgcaaatgaacagcctgcgtgccgaggacaccgcggtatattactgtgcgaaacggcatgcggctggggtttcgggtacttattttgactactggggtcagggaaccctggtcaccgtctcgagc

SEQ ID NO: 108 - DNA, 357 bp, Artificial Sequence
  DOM23h-802 nucleic acid
gaggtgcagctgttggagtctgggggaggcttggtacagcctggggggtccctgcgtctctcctgtgcagcctccggattcacctttagtgaggggacgatgtggtgggtccgccaggctccagggaagggtctagagtgggtctcagctattttggctgctggttctaatacatactacgcagactccgtgaagggccggttcaccatctcccgcgacaattccaagaacacgctgtatctgcaaatgaacagcctgcgtgccgaggacaccgcggtatattactgtgcgaaaaagaggcaggagcgggatgggtttgactactggggtcagggaaccctggtcaccgtctcgagc

SEQ ID NO: 109 - DNA, 357 bp, Artificial Sequence
  DOM23h-803 nucleic acid
gaggtgcagctgttggagtctgggggaggcttggtacagcctggggggtccctgcgtctctcctgtgcagcctccggattcacctttagtgctgggcggatgtggtgggtccgccaggctccagggaagggtctagagtgggtctcagcgattaatcgggatggtactaggacatactacgcagactccgtgaagggccggttcaccatctcccgtgacaattccaagaacacgctgtatctgcaaatgaacagcctgcgtgccgaggacaccgcggtatattactgtgcgaaacatgatgatggtcatggtaattttgactactggggtcagggaaccctggtcaccgtctcgagc

SEQ ID NO: 110 - DNA, 360 bp, Artificial Sequence
  DOM23h-813 nucleic acid
gaggtgcagctgttggagtctgggggaggcttggtacagcctggggggtccctgcgtctctcctgtgcagcctccggatccacctttacggatgataggatgtggtgggtccgccaggctccagggaagggtctagagtgggtctcagctattcagcctgatggtcatacgacatactacgcagactccgtgaagggccggttcaccatctcccgcgacaattccaagaacacgctgtatctgcaaatgaacagcctgcgtgccgaggacaccgcggtatattactgtgcggaacaggatgttaaggggtcgtcttcgtttgactactggggtcagggaaccctggtcaccgtctcgagc

SEQ ID NO: 111 - DNA, 357 bp, Artificial Sequence
  DOM23h-815 nucleic acid
gaggtgcagctgttggagtctgggggaggcttggtacagcctggggggtccctgcgtctctcctgtgcagcctccggattcacctttgcggaggatcggatgtggtgggtccgccaggctccagggaagggtctagagtgggtctcagctattgatcctcagggtcagcatacatactacgcagactccgtgaagggccggttcaccatctcccgcgacaattccaagaacacgctgtatctgcaaatgaacagcctgcgtgccgaggacaccgcggtatattactgtgcgaaacagtctactgggtctgctacgtctgactactggggtcagggaaccctggtcaccgtctcgagc

SEQ ID NO: 112 - DNA, 372 bp, Artificial Sequence
  DOM23h-828 nucleic acid
gaggtgcagctgttggagtctgggggaggcttggtacagcctggggggtccctgcgtctctcctgtgcagcctccggattcacctttatgagttataggatgtggtgggtccgccaggctccagggaagggtctagagtgggtctcagctatttctccgagtggtagtgatacatactacgcagactccgtgaagggccggttcaccatctcccgcgacaattccaagaacacgctgtatctgcaaatgaacagcctgcgtgccgaggacaccgcggtatattactgtgcgaaacaggtggtggagtattcgcgtactcataagggtgtgtttgactactggggtcagggaaccctggtcaccgtctcgagc

SEQ ID NO: 113 - DNA, 366 bp, Artificial Sequence
  DOM23h-830 nucleic acid
gaggtgcagctgttggagtctgggggaggcttggtacagcctggggggttcctgcgtctctcctgtgcagcctccggattcacctttgaggggtataggatgtggtgggtccgccaggctccagggaagggtctagagtgggtctcagctattgattctctgggtgatcgtacatactacgcagactccgtgaagggccggttcaccatctcccgcgacaattccaagaacacgctgtatctgcaaatgaacagtctgcgtgccgaggacaccgcggtatattactgtgcgaaacaggggcttacgcatcagtctccgagtacttttgactactggggtcagggaaccctggtcaccgtctcgagc

SEQ ID NO: 114 - DNA, 348 bp, Artificial Sequence
  DOM23h-831 nucleic acid
gaggtgcagctgttggagtctgggggaggcttggtacagcctggagggtccctgcgtctctcctgtgcagcctccggattcacctttgaggcgtataagatgacgtgggtccgccaggctccagggaagggtctggagtgggtctcatatattacgccgtctggtggtcagacatactacgcagactccgtgaagggccggttcaccatctcccgcgacaattccaagaacacgctgtatctgcaaatgaacagcctgcgtgccgaggacaccgcggtatattactgtgcgaaatatggttcgagttttgactactggggtcagggaaccctggtcaccgtctcgagc

SEQ ID NO: 115 - DNA, 357 bp, Artificial Sequence
  DOM23h-840 nucleic acid
gaggtgcagctgttggagtctgggggaggcttggtacagcctggggggtccctgcgtctctcctgtgcagcctccggattcacctttggggatggtcgtatgtggtgggtccgccaggctccagggaagggtctagagtgggtctcagctattgagggggcgggttcggatacatactacgcagactccgtgaagggccggttcaccatctcccgcgacaattccaagaacacgctgtatctgcaaatgaacagcctgcgtgccgaggacaccgcggtatattactgtgcgaaacaggcgtcgcggaattcgccgtttgactactggggtcaggggaccctggtcaccgtctcgagc

SEQ ID NO: 116 - DNA, 360 bp, Artificial Sequence
  DOM23h-842 nucleic acid
gaggtgcagctgttggagtctgggggaggcttggtacagcctggggggtccctgcgtctctcctgtgcagcctccggattcacctttgatgatagtgagatggcgtgggcccgccaggctccagggaagggtctagagtgggtctcacttattcggcgtaatggtaatgctacatactacgcagactccgtgaagggccggttcaccatctcccgcgacaattccaagaacacgctgtatctgcaaatgaacagcctgcgtgccgaggacaccgcggtatattactgtgcgaaagttacgaaggatcgttctgtgctttttgactactggggtcagggaaccctggtcaccgtctcgagc

SEQ ID NO: 117 - DNA, 360 bp, Artificial Sequence
  DOM23h-843 nucleic acid
gaggtgcagctgttggagtctgggggaggcttggtacagcctggggggtccctgcgtctctcctgtgcagcctccggattcacctttgatcaggatcggatgtggtgggtccgccaggctccagggaagggtctagagtgggtctcagctattgagagtggtggtcataggacatactacgcagactccgtgaagggccggttcaccatctcccgcgacaattccaagaacacgctgtatctgcaaatgaacagcctgcgtgccgaggacaccgcggtatattactgtgcgaaacagaatgagtcggggcgttcgggttttgactactggggtcagggaaccctggtcaccgtctcgagc

SEQ ID NO: 118 - DNA, 357 bp, Artificial Sequence
  DOM23h-850 nucleic acid
gaggtgcagctgttggagtctgggggaggcttggtacagcctggggggtccctgcgtctctcctgtgcagcctccggattcacctttgatgcggctaggatgtggtgggcccgccaggctccagggaagggtctagagtgggtctcagcgattgcggatattggtaatactacatactacgcagactccgtgaagggccggttcaccatctcccgcgacaattccaagaacacgctgtatctgcaaatgaacagcctgcgtgccgaggacaccgcggtatattactgtgcgaaacagtctggttcggaggatcattttgactactggggtcagggaaccctggtcaccgtctcgagc

SEQ ID NO: 119 - DNA, 363 bp, Artificial Sequence
  DOM23h-854 nucleic acid
gaggtgcagctgttggagtccgggggaggcttggtacagcctggggggtccctgcgtctctcctgtgcagcctccggattcacctttgctcaggatcggatgtggtgggtccgccaggctccagggaagggtctagagtgggtctcagctattagtggtagtggtggtagcacatactacgcagactccgtgaagggccggttcaccatctcccgcgacaattccaagaacacgctgtatctgcaaatgaacagcctgcgtgccgaggacaccgcggtatattactgtgcgaaacaggatttgcatggtactagttctttgtttgactactggggtcagggaaccctggtcaccgtctcgagc

SEQ ID NO: 120 - DNA, 360 bp, Artificial Sequence
  DOM23h-855 nucleic acid
gaggtgcagctgttggagtctgggggaggcttggtacagcctggggggtccctgcgtctctcctgtgcagcctccggattcacctttgagaatacgagtatgggttgggtccgccaggctccagggaagggtctagagtgggtctcacgtattgatcctaagggtagtcatacatactacgcagactccgtgaagggccggttcaccatctcccgcgacaattccaagaatacgctgtatctgcaaatgaacagcctgcgtgccgaggacaccgcggtatattactgtgcgaaacagcgtgagttgggtaagtcgcattttgactactggggtcagggaaccctggtcaccgtctcgagc

SEQ ID NO: 121 - DNA, 357 bp, Artificial Sequence
  DOM23h-865 nucleic acid
gaggtgcagctgttggagtctgggggaggcttggtacagcctggggggtccctgcgtctctcctgtgcagcctccggattcacctttcgtagttatgagatgacttgggtccgccaggctccagggaagggtctagagtgggtctcaaagattgatccttcgggtcgttttacatactacgcagactccgtgaagggccggttcaccatctcccgcgacaattccaagaacacgctgtatctgcaaatgaacagcctgcgtgccgaggataccgcggtatattactgtgcgaaaggtcggacggatcttcagctttttgactactggggtcagggaaccctggtcaccgtctcgagc

SEQ ID NO: 122 - DNA, 363 bp, Artificial Sequence
  DOM23h-866 nucleic acid
gaggtgcagctgttggagtctgggggaggcttggtacagcctggggggtccctgcgtctctcctgtgcagcctccggattcaccttttcgaattattggatgcggtgggcccgccaggctccagggaagggtctagagtgggtctcatatattactcctaagggtgatcatacatactacgcagactccgtgaagggccggttcaccatctcccgcgacaattccaagaacacgctgtatctgcaaatgaacagcctgcgtgccgaggacaccgcggtatattactgtgcggaatcgcttcataatgagcgtgttaagcattttgactactggggtcagggaaccctggtcaccgtctcgagc

SEQ ID NO: 123 - DNA, 363 bp, Artificial Sequence
  DOM23h-874 nucleic acid
gaggtgcagctgttggagtctgggggaggcttggtacagcctggggggtccctgcgtctctcctgtgcagcctccggattcacctttactagttatcgtatgtggtgggtccgccaggctccagggaagggtctagagtgggtctcagttattgattctactggttcggctacatactacgcagactccgtgaagggccggttcaccatctcccgcgacaattccaagaacacgctgtatctgcaaatgaacagcctgcgtgccgaggataccgcggtatattactgtgcgaaacagcaggctgggagtgcgatgggggagtttgactactggggtcagggaaccctggtcaccgtctcgagc

SEQ ID NO: 124 - DNA, 348 bp, Artificial Sequence
  DOM23h-883 nucleic acid
gaggtgcagctgttggagtctgggggaggcttggtacagcctggggggtccctgcgtctctcctgtgcagcctccggattcacctttgttaattatcgtatgtggtgggtccgccaggctccagggaagggtctagagtgggtctcagctattagtggtagtggtgataagacatactacgcagactccgtgaagggccggttcaccatctcccgcgacaattccaagaacacgctgtatctgcaaatgaacagcctgcgtgccgaggacaccgcggtatattactgtgcgaaacatgggctgtcgtttgactactggggtcagggaaccctggtcaccgtctcgagc

SEQ ID NO: 125 - DNA, 348 bp, Artificial Sequence
  DOM23h-903 nucleic acid
gaggtgcagctgttggagtctgggggaggcttggtacagcctggggggtccctgcgtctctcctgtgcagcctccggattcacctttaatgatatgaggatgtggtgggtccgccaggctccagggaagggtctagagtgggtctcagtgattaatgctgatggtaataggacatactacgcagactccgtgaagggccggttcaccatctcccgcgacaattccaagaacacgctgtatctgcaaatgaacagcctgcgtgccgaggataccgcggtatattactgtgcgaaagatgggctgccttttgactactggggtcagggaaccctggtcaccgtctcgagc

SEQ ID NO: 126 - DNA, 369 bp, Artificial Sequence
  DOM23m-4 nucleic acid
gaggtgcagctgttggagtctgggggaggcttggtacagcctggggggtccctgcgtctctcctgtgcagcctccggattcacctttacgacttatggtatgggttgggtccgccaggctccagggaagggtctagagtgggtctcatggattgagaagacgggtaataagacatactacgcagactccgtgaagggccggttcaccatctcccgcgacaattccaagaacacgctgtatctgcaaatgaacagcctgcgtgccgaggacaccgcggtatattactgtgcgaaagcggggaggcatattaaggtgcgttcgagggattttgactactggggtcagggaaccctggtcaccgtctcgagc

SEQ ID NO: 127 - DNA, 369 bp, Artificial Sequence
  DOM23m-29 nucleic acid
gaggtgcagctgttggagtctgggggaggcttggtacagcctggggggtccctgcgtctctcctgtgcagcctccggattcacctttaagaggtattctatgggttgggtccgccaggctccagggaagggtctagagtgggtctcagttattaatgatctgggtagtttgacatactacgcagactccgtgaagggccggttcaccatctcccgcgacaattccaagaacacgctgtatctgcaaatgaacagcctgcgtgccgaggacaccgcggtatattactgtgcgaaagggaatattagtatggtgaggccggggagttggtttgactactggggtcagggaaccctggtcaccgtctcgagc

SEQ ID NO: 128 - DNA, 369 bp, Artificial Sequence
  DOM23m-32 nucleic acid
gaggtgcagctgttggagtctgggggaggcttggtacagcctggggggtccctgcgtctctcctgtgcagcctccggattcaccttttttgagtatcctatgggttgggtccgccaggctccagggaagggtctagagtgggtctcagttattagtggggatggtcagcggacatactacgcagactccgtgaagggccggttcaccatctcccgcgacaattccaagaacacgctgtatctgcaaatgaacagcctgcgtgccgaggacaccgcggtatattactgtgcgaaaagtcatacggggactgtgaggcatctggagacgtttgactactggggtcagggaaccctggtcaccgtctcgagc

SEQ ID NO: 129 - DNA, 360 bp, Artificial Sequence
  DOM23m-62 nucleic acid
gaggtgcagctgttggagtctgggggaggcttggtacagcctggggggtccctgcgtctctcctgtgcagcctccggattcacctttggtcaggagagtatgtattgggtccgccaggctccagggaagggtctagagtgggtctcagctattagtggtagtggtggtagcacatactacgcagactccgtgaagggccggttcaccatctcccgcgacaattccaagaacacgctgtatctgcaaatgaacagcctgcgtgccgaggacaccgcggtatattactgtgcgaaaagtggtacgcggattaagcagggttttgactactggggtcagggaaccctggtcaccgtctcgagc

SEQ ID NO: 130 - DNA, 369 bp, Artificial Sequence
  DOM23m-71 nucleic acid
gaggtgcagctgttggagtctgggggaggcttggtacagcctggggggtccctgcgtctctcctgtgcagcctccggattcacctttatggattataggatgtattgggtccgccaggctccagggaagggtctagagtgggtctcagggattgatcctactggtttgcggacatactacgcagactccgtgaagggccggttcaccatctcccgcgacaattccaagaacacgctgtatctgcaaatgaacagcctgcgtgccgaggacaccgcggtatattactgtgcgaaaattaagtggggggagatggggagttataagacttttgactactggggtcagggaaccctggtcaccgtctcgagc

SEQ ID NO: 131 - DNA, 372 bp, Artificial Sequence
  DOM23m-72 nucleic acid
gaggtgcagctgttggagtctgggggaggcttggtacagcctggggggtccctgcgtctctcctgtgcagcctccggattcacctttatggattatgatatgagttgggtccgccaggctccagggaagggtctagagtgggtctcaatgattcgtgaggatggtggtaagacatactacgcagactccgtgaagggccggttcaccatctcccgcgacaattccaagaacacgctgtatctgcaaatgaacagcctgcgtgccgaggacaccgcggtatattactgtgcgaaagcgagggtgccttatcggcgtgggcatagggataattttgactactggggtcagggaaccctggtcaccgtctcgagc

SEQ ID NO: 132 - DNA, 360 bp, Artificial Sequence
  DOM23m-81 nucleic acid
gaggtgcagctgttggagtctgggggaggcttggtacagcctggggggtccctgcgtctctcctgtgcagcttccggattcacctttgagccggttattatggggtgggtccgccaggctccagggaagggtctagagtgggtctcagctattgaggcgcggggtggggggacatactacgcagactccgtgaagggccggttcaccatctcccgcgacaattccaagaacacgctgtatctgcaaatgaacagcctgcgtgccgaggacaccgcggtatattactgtgcgaaacctgggcggcatcttagtcaggattttgactactggggtcagggaaccctggtcaccgtctcgagc

SEQ ID NO: 133 - DNA, 357 bp, Artificial Sequence
  DOM23m-99 nucleic acid
gaggtgcagctgttggagtctgggggaggcttggtacagcctggggggtccctgcgtctctcctgtgcagcctccggattcacctttgatcggtatcgtatgatgtgggtccgccaggctccagggaagggtctagagtgggtctcaacgattgatcctgctggtatgcttacatactacgcagactccgtgaagggccggttcaccatctcccgcgacaattccaagaacacgctgtatctgcaaatgaacagcctgcgtgccgaggacaccgcggtatattactgtgcgaaaaggctggcttcgcggagtcattttgactactggggtcagggaaccctggtcaccgtctcgagc

SEQ ID NO: 134 - DNA, 357 bp, Artificial Sequence
  DOM23m-101 nucleic acid
gaggtgcagctgttggagtctgggggaggcttggtacagcctggggggtccctgcgtctctcctgcgcagcctccggattcaccttttctgagtatgatatggcttgggtccgccaggctccagggaagggtcttgagtgggtctcacggattcgttctgatggtgttaggacatactacgcagactccgtgaagggccggttcaccatctcccgcgacaattccaagaacacgctgtatctgcaaatgaacagcctgcgtgccgaggacaccgcggtatattactgtgcgaaagatcgtgctaagaatggttggtttgactactggggtcagggaaccctggtcaccgtctcgagc

SEQ ID NO: 135 - DNA, 357 bp, Artificial Sequence
  DOM23m-352 nucleic acid
gaggtgcagctgttggagtctgggggaggcttggtacagcctggggggtccctgcgtctctcctgtgcagcctccggattcacctttgataagtataagatggcttgggtccgccaggctccagggaagggtctggagtgggtctcacttatttttccgaatggtgttcctacatactacgcaaactccgtgaagggccggttcaccatctcccgcgacaattccaagaacacgctgtatctgcaaatgaacagcctgcgtgccgaggacaccgcggtatattactgtgcgaaatatagtggtcaggggcgggattttgactactggggtcagggaaccctggtcaccgtctcgagc

SEQ ID NO: 136 - DNA, 372 bp, Artificial Sequence
  DOM23h-271-21 nucleic acid
gaggtgcagctgttggagtctgggggaggcttggtacagcctggggggtccctgcgtctctcctgtgcagcctccggattcacctttaccgagtataggatgtggtgggtccgccaggctccggggaagggtctcgagtgggtctcagcgattgagccgattggtaatcgtacatactacgcagactccgtgaagggccggttcaccatctcccgcgacaattccaagaacacgctgtatctgcaaatgaacagcctgcgtgccgaggataccgcggtatattactgtgcgaaacagatgccgggccggaagtggacggccaagttccgctgggactactggggtcagggaaccctggtcatcgtctcgagc

SEQ ID NO: 137 - DNA, 372 bp, Artificial Sequence
  DOM23h-271-22 nucleic acid
gaggtgcagctgttggagtctgggggaggcttggtacagcctggggggtccctgcgtctctcctgtgcagcctccggattcacctttacggagtataggatgtggtgggtccgccaggctccggggaagggtctcgagtgggtctcagcgattgagccgattggtaatcgtacatactacgcagactccgtgaagggccggttcaccatctcccgcgacaattccaagaacacgctgtatctgcaaatgaacagcctgcgtgccgaggataccgcggtatattactgtgcgaaacagatgcccggccagaagtggatggccaagtcccgcttcgactactggggtcagggaaccctggtcaccgtctcgagc

SEQ ID NO: 138 - DNA, 372 bp, Artificial Sequence
  DOM23h-271-27 nucleic acid
gaggtgcagctgttggagtctgggggaggcttggtacagcctggggggtccctgcgtctctcctgtgcagcctccggattcacctttacggagtataggatgtggtgggtccgccaggctccagggaagggtctcgagtgggtctcagcgattgagccgattggtcagaagacatactacgcagactccgtgaagggccggttcaccatctcccgcgacaattccaagaacacgctgtatctgcaaatgaacagcctgcgtgccgaggataccgcggtatattactgtgcgaaacagattccggggcgtaagtggactgctaattcgcggtttgactactggggtcagggaaccctggtcatcgtctcgagc

SEQ ID NO: 139 - DNA, 372 bp, Artificial Sequence
  DOM23h-271-101 nucleic acid
gaggtgcagctgttggagtctgggggaggcttggtacagcctggggggtccctgcgtctctcctgtgcagcctccggattcacctttacggagtataggatgtggtgggtccgccaggctccggggaagggtctcgagtgggtctcagcgattgagccgattggtaatcgtacatactacgcagactccgtgaagggccggttcaccatctcccgcgacaattccaagaacacgctgtatctgcaaatgaacagcctgcgtgccgaggataccgcggtatattactgtgcgaaacagattccggggcgtaagtggactgctaatggtcgtaaggactactggggtcagggaaccctggtcaccgtctcgagc

SEQ ID NO: 140 - DNA, 372 bp, Artificial Sequence
  DOM23h-271-102 nucleic acid
gaggtgcagctgttggagtctgggggaggcttggtacagcctggggggtccctgcgtctctcctgtgcagcctccggatccacctttacggagtataggatgtggtgggtccgccaggctccggggaagggtctcgagtgggtctcagcgattgagccgattggtcataggacatactacgcagactccgtgaagggccggttcaccatctcccgcgacaattccaagaacacgctgtatctgcaaatgaacagcctgcgtgccgaggataccgcggtatattactgtgcgaaacagattccggggcgtaagtggactgctaattcgcggtttgactactggggtcagggaaccctggtcaccgtctcgagc

SEQ ID NO: 141 - DNA, 372 bp, Artificial Sequence
  DOM23h-271-105 nucleic acid
gaggtgcagctgttggagtctgggggaggcttggtacagcctggggggtccctgcgtctctcctgtgcagcctccggattcacctttacggagtataggatgtggtgggtccgccaggctccggggaagggtctcgagtgggtctcagcgattgagccgattggtaatcgtacatactacgcagactccgtgaagggccggttcaccatctcccgcgacaattccaagaacacgctgtatctgcaaatgaacagcctgcgtgccgaggataccgcggtatattactgtgcgaaacagattccggggcagcggtggactggtaattcgcggtttgactactggggtcagggaaccctggtcaccgtctcgagc

SEQ ID NO: 142 - DNA, 372 bp, Artificial Sequence
  DOM23h-271-106 nucleic acid
gaggtgcagctgttggagtctgggggaggcttggtacagcctggggggtccctgcgtctctcctgtgcagcctccggattcacctttacggagtataggatgtggtgggtccgccaggctccggggaagggtctcgagtgggtctcagcgattgagccgattggtaatcgtacatactacgcagactccgtgaagggccggttcaccatctcccgcgacaattccaagaacacgctgtatctgcaaatgaacagcctgcgtgccgaggataccgcggtatattactgtgcgaaacagtttccggggcgtaagtggactgctaattcgcggtctgactactggggtcagggaaccctggtcaccgtctcgagc

SEQ ID NO: 143 - DNA, 372 bp, Artificial Sequence
  DOM23h-271-114 nucleic acid
gaggtgcagctgttggagtctgggggaggcttggtacagcctggggggtccctgcgtctctcctgtgcagcctccggattcacctttacggagtataggatgtggtgggtccgccaggctccggggaagggtctcgagtgggtctcagcgattgagccgattggtaatcgtacatactacgcagactccgtgaagggccggttcaccatctcccgcgacaattccaagaacacgctgtatctgcaaatgaacagcctgcgtgccgaggataccgcggtatattactgtgcgaaacagattccggggcgtaagggaactgctaattcgcggtttgactactggggtcagggaaccctggtcaccgtctcgagc

SEQ ID NO: 144 - DNA, 372 bp, Artificial Sequence
  DOM23h-271-39 nucleic acid
gaggtgcagctgttggagtctgggggaggcttggtacagcctggggggtccctgcgtctctcctgtgcagcctccggattcacctttacggagtataggatgtggtgggtccgccaggctccggggaagggtctcgagtgggtctcagcgattgagccgattggtaatcgtacatactacgcacgctccgtggacggccggttcaccatctcccgcgacaattccaagaacacgctgtatctgcaaatgaacagcctgcgtgccgaggataccgcggtatattactgtgcgaaacagatgcccggccagaagtggatggccaagtcccgcttcgactactggggtcagggaaccctggtcaccgtctcgagc

SEQ ID NO: 145 - DNA, 372 bp, Artificial Sequence
  DOM23h-271-40 nucleic acid
gaggtgcagctgttggagtctgggggaggcttggtacagcctggggggtccctgcgtctctcctgtgcagcctccggattcacctttacggagtataggatgtggtgggtccgccaggctccggggaagggtctcgagtgggtctcagcgattgagccgattggtaatcgtacatactacgcacgctccgtgttcggccggttcaccatctcccgcgacaattccaagaacacgctgtatctgcaaatgaacagcctgcgtgccgaggataccgcggtatattactgtgcgaaacagatgcccggccagaagtggatggccaagtcccgcttcgactactggggtcagggaaccctggtcaccgtctcgagc

SEQ ID NO: 146 - DNA, 360 bp, Artificial Sequence
  DOM23h-855-21 nucleic acid
gaggtgcagctgttggagtctgggggaggcttggtacagcctggggggtccctgcgtctctcctgtgcagcctccggattcacctttgagaatacgagtatgggttgggtccgccaggctccagggaagggtctagagtgggtctcacgtattgatcctaagggtagtcatacatactacacagactccgtgaagggccggttcaccatctcccgcgacaattccaagaatacgctgtatctgcaaatgaacagcctgcgtgccgaggacaccgcggtatattactgtgcgaaacagcgtgagttgggtaagtcgtattttgactactggggtcagggaaccctggtcaccgtctcgagc

SEQ ID NO: 147 - DNA, 360 bp, Artificial Sequence
  DOM23h-843-13 nucleic acid
gaggtgcagctgttggagtctgggggaggcctggtacagcctggggggtccctgcgtctctcctgtgcagcctccggattcacctttgatcaggatcggatgtggtgggtccgccaggccccagggaagggtctagagtgggtctcagctattgagagtggtggtcataggacatactacgcagactccgtgaagggccggttcaccatctcccgcgacaattccaagaacacgctgtatctgcaaatgaacagcctgcgtgccgaggacaccgcggtatattactgtgcgaatcagaataagtcggggcgttcgggttttgactactggggtcagggaaccctggtcaccgtctcgagc

SEQ ID NO: 148 - DNA, 366 bp, Artificial Sequence
  DOM23h-439-20 nucleic acid
gaggtgcagctgttggagtctgggggaggcttggtacagcctggggggtccctgcgtctctcctgtgcagcctccggattcacctttgggacggagcagatgtggtgggtccgccaggctccagggaagggtctagagtttgtctcacgtattgattcgcctggtgggaggacatactacgcagactccgtgaagggccggttcaccatctcccgcgacaattccaagaacacgctgtatctgcaaatgaacagcctgcgtgccgaggacaccgcggtatattactgtgcgaaacggcatgcggctggggtttcgggtacttattttgactactggggtcagggaaccctggtcaccgtctcgagc

SEQ ID NO: 149 - DNA, 372 bp, Artificial Sequence
  DOM23h-271-50 nucleic acid
gaggtgcagctgttggagtctgggggaggcttggtacagcctggggggtccctgcgtctctcctgtgcagcctccggatccacctttacggagtataggatgtggtgggtccgccaggctccggggaagggtctcgagtgggtctcagcgattgagccgattggtcataggacatactacgcagactccgtgaagggccggttcaccatctcccgcgacaattccaagaacacgctgtatctgcaaatgaacagcctgcgtgccgaggataccgcggtatattactgtgcgaaacaggcgcccggcgagaagtggctcgcccggggccgcttggactactggggtcagggaaccctggtcaccgtctcgagc

SEQ ID NO: 150 - DNA, 366 bp, Artificial Sequence
  DOM23h-439-25 nucleic acid
gaggtgcagctgttggagtctgggggaggcttggtacagcctggggggtccctgcgtctctcctgtgcagcctccggattcacctttgggacggagcagatgtggtgggtccgccaggctccagggaagggtctagagtttgtctcacgtattgattcgcctggtgggaggacatactacgcagactccgtgaagggccggttcaccatctcccgcgacaattccaagaacacgctgtatctgcaaatgaacagcctgcgtgccgaggacaccgcggtatattactgtgcgaaacggcgacccacgggggtgtccgggacgttttatgactactggggtcagggaaccctggtcaccgtctcgagc

SEQ ID NO: 151 - DNA, 372 bp, Artificial Sequence
  DOM23h-271-123 nucleic acid
gaggtgcagctgttggagtctgggggaggcttggtacagcctggggggtccctgcgtctctcctgtgcagcctccggatccacctttacggagtataggatgtggtgggtccgccaggctccggggaagggtctcgagtgggtctcagcgattgagccgattggtcataggacatactacgcagactccgtgaagggccggttcaccatctcccgcgacaattccaagaacacgctgtatctgcaaatgaacagcctgcgtgccgaggataccgcggtatattactgtgcgaaacaggcgcccggcgagaagtgggcgaggcggtgggatttggactactggggtcagggaaccctggtcaccgtctcgagc

SEQ ID NO: 152 - DNA, 366 bp, Artificial Sequence
  DOM23h-439-35 nucleic acid
gaggtgcagctgttggagtctgggggaggcttggtacagcctggggggtccctgcgtctctcctgtgcagcctccggattcacctttgggaccgatcagatgtggtgggtccgccaggctccagggaagggtctagagtttgtctcacgcattgattcccccggtgggcggacatactacgcaaactccgtgaagggccggttcaccatctcccgcgacaattccaagaacacgctgtatctgcaaatgaacagcctgcgtgccgaggacaccgcggtatattactgtgcgaaacggcagccggcgggggtgtcggggaagtacgttgactactggggtcagggaaccctggtcaccgtctcgagc

SEQ ID NO: 153 - DNA, 372 bp, Artificial Sequence
  DOM23h-271-129 nucleic acid
gaggtgcagctgttggagtctgggggaggcttggtacagcctggggggtccctgcgtctctcctgtgcagcctccggatccacctttacggagtataggatgtggtgggtccgccaggctccggggaagggtctcgagtgggtctcagcgattgagccgattggtcataggacatactacgcagactccgtgaagggccggttcaccatctcccgcgacaattccaagaacacgctgtatctgcaaatgaacagcctgcgtgccgaggataccgcggtatattactgtgcgaaacaggcgcccaatcagaggtatgttgcccggggccgcttggactactggggtcagggaaccctggtcaccgtctcgagc

SEQ ID NO: 154 - DNA, 369 bp, Artificial Sequence
  DOM23h-439-40 nucleic acid
gaggtgcagctgttggagtctgggggaggcttggtacagcctggggggtccctgcgtctctcctgtgcagcctccggattcacctttgggacggagcagatgtggtgggtccgccaggctccagggaagggtctagagtttgtctcacgtattgattcgcctggtgggaggacatactacgcagactccgtgaagggccggttcaccatctcccgcgacaattccaagaacacgctgtatctgcaaatgaacagcctgcgtgccgaggacaccgcggtatattactgtgcgaaacggcgacccacgggggtgtccgggacgttttatgactactggggtcagggaaccctggtcaccgtctcgagcgcg

SEQ ID NO: 155 - DNA, 369 bp, Artificial Sequence
  DOM23h-439-41 nucleic acid
gaggtgcagctgttggagtctgggggaggcttggtacagcctggggggtccctgcgtctctcctgtgcagcctccggattcacctttgggacggagcagatgtggtgggtccgccaggctccagggaagggtctagagtttatttcacgtattgattcgcctggtgggaggacatactacgcagactccgtgaagggccggttcaccatctcccgcgacaattccaagaacacgctgtatctgcaaatgaacagcctgcgtgccgaggacaccgcggtatattactgtgcgaaacggcgacccacgggggtgtccgggacgttttatgactactggggtcagggaaccctggtcaccgtctcgagcgcg

SEQ ID NO: 156 - DNA, 369 bp, Artificial Sequence
  DOM23h-439-42 nucleic acid
gaggtgcagctgttggagtctgggggaggcttggtacagcctggggggtccctgcgtctctcctgtgcagcctccggattcacctttgggacggagcagatgtggtgggtccgccaggctccagggaagggtctagagtttgtctcacgtattgattcgcctggtgggaggacatactacgcaaactccgtgaagggccggttcaccatctcccgcgacaattccaagaacacgctgtatctgcaaatgaacagcctgcgtgccgaggacaccgcggtatattactgtgcgaaacggcgacccacgggggtgtccgggacgttttatgactactggggtcagggaaccctggtcaccgtctcgagcgcg

SEQ ID NO: 157 - DNA, 369 bp, Artificial Sequence
  DOM23h-439-43 nucleic acid
gaggtgcagctgttggagtctgggggaggcttggtacagcctggggggtccctgcgtctctcctgtgcagcctccggattcacctttgggacggagcagatgtggtgggtccgccaggctccagggaagggtctagagtttgtctcacgtattgattcgcctggtgggaggacatactacgcagactccgtgcgtggccggttcaccatctcccgcgacaattccaagaacacgctgtatctgcaaatgaacagcctgcgtgccgaggacaccgcggtatattactgtgcgaaacggcgacccacgggggtgtccgggacgttttatgactactggggtcagggaaccctggtcaccgtctcgagcgcg

SEQ ID NO: 158 - DNA, 369 bp, Artificial Sequence
  DOM23h-439-44 nucleic acid
gaggtgcagctgttggagtctgggggaggcttggtacagcctggggggtccctgcgtctctcctgtgcagcctccggattcacctttgggacggagcagatgtggtgggtccgccaggctccagggaagggtctagagtttgtctcacgtattgattcgcctggtgggaggacatactacgcaaactccgtgcgtggccggttcaccatctcccgcgacaattccaagaacacgctgtatctgcaaatgaacagcctgcgtgccgaggacaccgcggtatattactgtgcgaaacggcgacccacgggggtgtccgggacgttttatgactactggggtcagggaaccctggtcaccgtctcgagcgcg

SEQ ID NO: 159 - DNA, 375 bp, Artificial Sequence
  DOM23h-271-130 nucleic acid
gaggtgcagctgttggagtctgggggaggcttggtacagcctggggggtccctgcgtctctcctgtgcagcctccggatccacctttacggagtataggatgtggtgggtccgccaggctccggggaagggtctcgagtgggtctcagcgattgagccgattggtcataggacatactacgcagactccgtgaagggccggttcaccatctcccgcgacaattccaagaacacgctgtatctgcaaatgaacagcctgcgtgccgaggataccgcggtatattactgtgcgaaacaggcgcccggcgagaagtgggcgaggcggtgggatttggactactggggtcagggaaccctggtcaccgtctcgagcgcg

SEQ ID NO: 160 - DNA, 375 bp, Artificial Sequence
  DOM23h-271-131 nucleic acid
gaggtgcagctgttggagtctgggggaggcttggtacagcctggggggtccctgcgtctctcctgtgcagcctccggatccacctttacggagtataggatgtggtgggtccgccaggctccggggaagggtctcgagtgggtctcagcgattgagccgattggtcataggacatactacgcaaactccgtgaagggccggttcaccatctcccgcgacaattccaagaacacgctgtatctgcaaatgaacagcctgcgtgccgaggataccgcggtatattactgtgcgaaacaggcgcccggcgagaagtgggcgaggcggtgggatttggactactggggtcagggaaccctggtcaccgtctcgagcgcg

SEQ ID NO: 161 - DNA, 375 bp, Artificial Sequence
  DOM23h-271-132 nucleic acid
gaggtgcagctgttggagtctgggggaggcttggtacagcctggggggtccctgcgtctctcctgtgcagcctccggatccacctttacggagtataggatgtggtgggtccgccaggctccggggaagggtctcgagtgggtctcagcgattgagccgattggtcataggacatactacgcagactccgtgcgtggccggttcaccatctcccgcgacaattccaagaacacgctgtatctgcaaatgaacagcctgcgtgccgaggataccgcggtatattactgtgcgaaacaggcgcccggcgagaagtgggcgaggcggtgggatttggactactggggtcagggaaccctggtcaccgtctcgagcgcg

SEQ ID NO: 162 - DNA, 375 bp, Artificial Sequence
  DOM23h-271-133 nucleic acid
gaggtgcagctgttggagtctgggggaggcttggtacagcctggggggtccctgcgtctctcctgtgcagcctccggatccacctttacggagtataggatgtggtgggtccgccaggctccggggaagggtctcgagtgggtctcagcgattgagccgattggtcataggacatactacgcaaactccgtgcgtggccggttcaccatctcccgcgacaattccaagaacacgctgtatctgcaaatgaacagcctgcgtgccgaggataccgcggtatattactgtgcgaaacaggcgcccggcgagaagtgggcgaggcggtgggatttggactactggggtcagggaaccctggtcaccgtctcgagcgcg

SEQ ID NO: 163 - DNA, 375 bp, Artificial Sequence
  DOM23h-271-134 nucleic acid
gaggtgcagctgttggagtctgggggaggcttggtacagcctggggggtccctgcgtctctcctgtgcagcctccggatccacctttacggagtataggatgtggtgggtccgccaggctccggggaagggtctcgagtggatttcagcgattgagccgattggtcataggacatactacgcagactccgtgaagggccggttcaccatctcccgcgacaattccaagaacacgctgtatctgcaaatgaacagcctgcgtgccgaggataccgcggtatattactgtgcgaaacaggcgcccggcgagaagtgggcgaggcggtgggatttggactactggggtcagggaaccctggtcaccgtctcgagcgcg

SEQ ID NO: 164 - DNA, 375 bp, Artificial Sequence
  DOM23h-271-135 nucleic acid
gaggtgcagctgttggagtctgggggaggcttggtacagcctggggggtccctgcgtctctcctgtgcagcctccggatccacctttacggagtataggatgtggtgggtccgccaggctccggggaagggtctcgagtgggtctcagcgattgagccgattggtcataggacatactacgcagactccgtgaagggccggttcaccatctcccgcgacaattccaagaacacgctgtatctgcaaatgaacagcctgcgtgccgaggataccgcggtatattactgtgcgaaacaggcgcccaatcagaggtatgttgcccggggccgcttggactactggggtcagggaaccctggtcaccgtctcgagcgcg

SEQ ID NO: 165 - DNA, 375 bp, Artificial Sequence
  DOM23h-271-136 nucleic acid
gaggtgcagctgttggagtctgggggaggcttggtacagcctggggggtccctgcgtctctcctgtgcagcctccggatccacctttacggagtataggatgtggtgggtccgccaggctccggggaagggtctcgagtgggtctcagcgattgagccgattggtcataggacatactacgcaaactccgtgaagggccggttcaccatctcccgcgacaattccaagaacacgctgtatctgcaaatgaacagcctgcgtgccgaggataccgcggtatattactgtgcgaaacaggcgcccaatcagaggtatgttgcccggggccgcttggactactggggtcagggaaccctggtcaccgtctcgagcgcg

SEQ ID NO: 166 - DNA, 375 bp, Artificial Sequence
  DOM23h-271-137 nucleic acid
gaggtgcagctgttggagtctgggggaggcttggtacagcctggggggtccctgcgtctctcctgtgcagcctccggatccacctttacggagtataggatgtggtgggtccgccaggctccggggaagggtctcgagtgggtctcagcgattgagccgattggtcataggacatactacgcaaactccgtgcgtggccggttcaccatctcccgcgacaattccaagaacacgctgtatctgcaaatgaacagcctgcgtgccgaggataccgcggtatattactgtgcgaaacaggcgcccaatcagaggtatgttgcccggggccgcttggactactggggtcagggaaccctggtcaccgtctcgagcgcg

SEQ ID NO: 167 - DNA, 369 bp, Artificial Sequence
  DOM23h-439-47 nucleic acid
gaggtgcagctgttggagtctgggggaggcttggtacagcctggggggtccctgcgtctctcctgtgcagcctccggattcacctttgggacggagcagatgtggtgggtccgccaggctccagggaagggtctagagtttatttcacgtattgattcgcctggtgggaggacatactacgcaaactccgtgaagggccggttcaccatctcccgcgacaattccaagaacacgctgtatctgcaaatgaacagcctgcgtgccgaggacaccgcggtatattactgtgcgaaacggcgacccacgggggtgtccgggacgttttatgactactggggtcagggaaccctggtcaccgtctcgagcgcg

SEQ ID NO: 168 - DNA, 369 bp, Artificial Sequence
  DOM23h-439-48 nucleic acid
gaggtgcagctgttggagtctgggggaggcttggtacagcctggggggtccctgcgtctctcctgtgcagcctccggattcacctttgggacggagcagatgtggtgggtccgccaggctccagggaagggtctagagtttatttcacgtattgattcgcctggtgggaggacatactacgcaaactccgtgcgtggccggttcaccatctcccgcgacaattccaagaacacgctgtatctgcaaatgaacagcctgcgtgccgaggacaccgcggtatattactgtgcgaaacggcgacccacgggggtgtccgggacgttttatgactactggggtcagggaaccctggtcaccgtctcgagcgcg